SEQUENCE LISTING (9 sequences)

SEQ ID NO: 1 - DNA, 33 bp, Artificial Sequence
  Primer
ggcccatggatgaaaaatataactactttcatt

SEQ ID NO: 2 - DNA, 30 bp, Artificial Sequence
  Primer
ggcccatggatgaataaagtaaaatgttat

SEQ ID NO: 3 - DNA, 27 bp, Artificial Sequence
  Primer
ggcggatccgttttccatactgattgc

SEQ ID NO: 4 - DNA, 30 bp, Artificial Sequence
  Primer
ggcggatcccaggatgctaaaccagtagag

SEQ ID NO: 5 - DNA, 30 bp, Artificial Sequence
  Primer
ggcctcgagctaatagcacccggtacaagc

SEQ ID NO: 6 - DNA, 27 bp, Artificial Sequence
  Linker
ggatccgaattcgagctccgtcgaccg

SEQ ID NO: 7 - protein, 9 aa, Artificial Sequence
  Linker
GSEFELRRP

SEQ ID NO: 8 - DNA, 12 bp, Artificial Sequence
  Linker
ggatccggtacc

SEQ ID NO: 9 - protein, 4 aa, Artificial Sequence
  Linker
GSGT

(19) United States
(12) Patent Application Publication
(10) Pub. No.: US 2009/0017056 A1
(43) Pub. Date: Jan. 15, 2009

(54) SKIN IMMUNIZATION USING LT-STA FUSION PROTEINS

(75) Inventors: Jian-Hui Tian, Gaithersburg, MD (US); Gregory M. Glenn, Gaithersburg, MD (US); Larry R. Ellingsworth, Gaithersburg, MD (US)

Correspondence Address: MORGAN LEWIS & BOCKIUS LLP, 1111 PENNSYLVANIA AVENUE NW, WASHINGTON, DC, 20004, US

(73) Assignee: IOMAI CORPORATION, Gaithersburg, MD (US)

(21) Appl. No.: 11/629,620

(22) PCT Filed: Jun. 15, 2005
(86) PCT No.: PCT/US2005/021001

§ 371 Date: Sep. 25, 2008

Related U.S. Application Data
(60) Provisional application No. 60/579,264, filed on Jun. 15, 2004.

(51) Int. Cl. A61K 39/02; C07K 19/00

(52) U.S. Cl. 424/192.1; 530/350

(57) ABSTRACT

This invention includes fusion proteins comprising a bacterial ADP-ribosylating exotoxin (bARE), or a variant or portion thereof, fused to a STa exotoxin, or a portion or variant thereof. Optionally, the exotoxins are fused via a peptide linker. The invention also includes compositions formulated for transcutaneous immunizations and/or induction of an immune response by epicutaneous administration comprising an effective amount of a fusion protein comprising a bacterial ADP-ribosylating exotoxin fused to a STa exotoxin. Optionally, the exotoxins are fused via a peptide linker.

### RELATED APPLICATIONS

This application claims the benefit of U.S. provisional application 60/579,264, filed Jun. 15, 2004, which is incorporated herein by reference in its entirety.

### FIELD OF THE INVENTION

The invention is in the field of compositions, formulations and methods of treatment comprising fusion proteins.

### BACKGROUND OF THE INVENTION

Diarrhea caused by enterotoxigenic Escherichia Coli (ETEC) is a disease associated with significant morbidity and mortality, particularly in children, in areas of the world where fecal contamination of food and water occurs. ETEC diarrhea is most closely associated with epithelial cell binding of either heat labile enterotoxin (LT), an 80 kDa protein, or by heat stable enterotoxin (ST), an 19 mer polypeptide toxin, or both and subsequent dysregulation of fluid homeostatis at the level of the intestinal epithelium. ETEC is second only to rota virus as the cause of severe dehydrating diarrhea in young children throughout the world and can be isolated from more than half of the children aged 2-3 years in endemic areas. It is estimated that ETEC causes more than 600 million cases of diarrhea per year and more that 400,000 deaths in children less than 5 years of age. ETEC is also the most common cause of Traveler's diarrhea in civilian and military populations. During the Persian Gulf War in 1990-91, diarrhea for any cause was reported by 57% of the United States troops stationed in Saudi Arabia, and 20% reported lost duty time due to illness. ETEC and shigella were the predominant causes of diarrhea among U.S. troops during deployment (Wolf et al. (1993) J. Clin. Microbiol. 31, 851-856; Hyms et al. (1991) N. Engl. J. Med. 325, 1423-1428). ETEC is also one of the main causes of food borne disease in developing countries (Todd (1997) World Health Stat. Q, 50, 30-50) and is an important cause of waterborne outbreaks of diarrheal disease (Huerta, et al. (2000) Infection 28, 267-271; Daniels et al. (2000) J. Infect. Dis. 181, 1491-1495).

Measures to avoid Traveler's diarrhea include hygienic measures that prevent the consumption of food or water contaminated with ETEC, however these hygienic measures are difficult to maintain during travel. Traveler's diarrhea is usually treated with oral antibiotics, rehydration and intestinal anti-motility agents. Antibiotic prophylaxis against ETEC Traveler's diarrhea has been tested and shown to be effective, however, drug resistance of ETEC against multiple antibiotics has been documented since the early 1980's and continues to be an issue of growing concern (Jiang et al. (2000) J. Infect Dis. 181, 779-782).

Experimental ETEC infection induces an immune response that protects against diseases on subsequent exposure and repeated “natural” exposure lead to acquired protective immunity (ETEC and enteric vaccines, Eds. Jong E, Traveler's Vaccines, Longon: BC Decker, Inc., 2004). This suggests that a vaccine is a possible solution, yet currently there are no licensed ETEC vaccines.

Vaccine development strategies for ETEC have recently focused on generating immune responses that prevent initial colonization of the bacteria in the gut. These strategies depend on the identification of prevalent colonization factor (CF) antigens and the identification of a subset of candidate antigens that could be developed as vaccine targets. A variety of CF antigens have been proposed as vaccine targets into early stage evaluation. However, the multiplicity of antigen targets, the regional nature of their prevalence, and changing patterns of prevalence suggest that the resources needed to clone, express, and optimize immune responses to a multivalent platform prior to evaluation of their relevance to protection may be extremely difficult. Each antigen would require proof of contribution to efficacy and a multivalent vaccine would finally need to be tested. Additionally, no direct evidence exists that immune responses to CFs will block adhesion, although passive oral antibody was able to protect against challenge. Protection by milk immunoglobulin concentrate against oral challenge with enterotoxigenic Escherichia coli showed some protection (Tacket et al. (1988) N. Engl. J. Med. 318, 1240-1243), but that result that was not confirmed by a later study (Tacket et al. (1999) J. Infect. Dis. 180, 2056-2059).

Thus, an effective neutralizing immunity to the two toxins that cause ETEC could simplify a development program focused on one or two antigens and provide complete strain coverage against ETEC disease. However, the use of these toxins presents difficulty in delivery as native antigens due to their toxicities in various settings. Bacterial ADP-ribosylating exotoxins (bAREs) are known to be highly toxic when injected or given systemically. For example, intradermal injections have been shown to induce persistent nodules when LT is included as the adjuvant (Guy et al. (1999) Vaccine 17, 1130-1135), cause unacceptable inflammation when injected into tissues, been implicated in Bell's palsy after intranasal use, and can cause diarrhea if taken orally. Strategies to modify LT or use the B subunit for oral use have lead to attenuation of immune responses although more recent mutants appear to have both potent immunogenicity as well as improved safety profiles. Notably, an oral cholera vaccine containing the cholera toxin B subunit has shown protection in field studies against ETEC (Clements et al. (1990) Lancet 335, 270-273) but protection appears to be short lived and not robust.

Recently, cholera toxin has been shown to be immunogenic, acting as both antigen and adjuvant, when placed on the skin but without any resulting local or systemic side effects. This lack of reactogenicity when cholera toxin was placed on the skin for transcutaneous immunization was surprising. It was not obvious prior to our studies that cholera toxin or other ADP-ribosylating exotoxins would be useful for transcutaneous immunization. See related U.S. application Ser. Nos. 08/896,085 and 09/311,720; and, U.S. Pat. No. 5,910,306, all incorporated by reference herein in their entireties.

Our studies have shown that bovine serum albumin (BSA), not highly immunogenic by itself when epicutaneously applied to the skin, can induce a strong immune response when placed on the skin with CT. The Langerhans cell population underlying the site of application is a preferred antigen presenting cell (APC) for activation, differentiation and delivering antigen to the immune system. Adjuvant may act on the APC directly, or through cognate lymphocytes specifically recognizing antigen. The induction of mucosal immunity and immunoprotection with the present invention would not have been expected by the art prior to the cited disclosures.

In addition, related U.S. application Ser. Nos. 08/749,164 (now U.S. Pat. No. 5,910,164), 08/896,085, and 09/311,720, incorporated by reference herein in their entireties, also show that using a wide variety of ADP-ribosylating exotoxins such as, heat-labile enterotoxin from E. coli (LT), Pseudomonas exotoxin A (ETA), and pertussis toxin (PT), can elicit a vigorous immune response to epicutaneous application which is highly reproducible. Moreover, when such skin-active adjuvants were applied along with a separate antigen (e.g., bovine serum albumin or diphtheria toxoid), systemic and mucosal antigen-specific immune responses could be elicited.

It has also been shown that as a vaccine antigen, LT is immunogenic and safe when administered topically. In fact, the safe use of LT when administered topically via the skin has been established in humans. To date over 1,500 volunteers have been topically treated with LT at very high doses (500 μg). A double blind, placebo controlled safety trial has been conducted without any serious adverse side effects. As a potent adjuvant and an immunogen, LT has proven to be safe when administered via the skin.

However, because of its small size Stable Toxin (ST) is poorly immunogenic. ST is one of the major causes of ETEC. Thus, a vaccine that has a ST as an immunogenic component would greatly increase the scope of protection for Traveler's diarrhea and other ETEC caused illnesses. However, the immunogenicity of ST is improved when coupled as a hapten to a carrier molecule such asalbumin. Conjugation strategies led to animal protection data (Klipstein et al. (1982) Infect. Immun. 37, 550-557) but apparently these studies did not lead to full vaccine development, in part due to the debate and conflicting data on the role of toxin based immunity.

There have also been a number of publications that have disclosed LT-STa fusion proteins for use in vaccination of mammals to prevent Traveler's disease (see, Cardenas et al. (1993) Infect. Immun. 61, 4629-4636; Clements (1990) Infect. Immun. 58, 1159-1166). These earlier studies have shown that LT-proSTa fusion proteins are immunogenic and do result in the generation of toxin neutralizing antibodies to STa and to LT. However, the immunization methods were with repeated doses administered by intraperitoneal injection and/or peroral route. Although these investigations demonstrate the increased immunity of STa, the route of immunization is not practical for a human vaccine. In addition, in most cases, no hybrid protein with properly folded STa joined covalently to the carrier protein was both extracellularly secreted and fully active (Batisson et al. (2000) Infect. Immun. 68, 4064-4074). Thus, an alternative strategy, disclosed herein, incorporates an amino acid linker between bARE, or fragments and/or subunits thereof, and STa.

Furthermore, related U.S. application Ser. Nos. 09/257,188, 60/128,370, and 09/309,881, incorporated by reference herein in their entireties, disclose penetration enhancers (e.g., removal of superficial layers above the dermis, micropenetration to above the dermis), dry formulations, and targeting of complexed antigen in the context of transcutaneous immunization, respectively, which may enhance transcutaneous immunization comprising LT-STa fusion proteins.

Thus, there is a need for an efficient and well tolerated vaccine against ETEC. Topical immunization is a new route, which we have shown to be safe and effective and without the side effects associated with immunizing with enterotoxins.

### SUMMARY OF THE INVENTION

The present invention provides fusion proteins comprising a bacterial ADP-ribosylating exotoxin (bARE), or a variant or portion thereof, fused to a STa exotoxin, or a portion or variant thereof, wherein said exotoxins are fused via a peptide linker. The invention also provides for compositions formulated for transcutaneous immunizations and/or induction of an immune response comprising an effective amount of a fusion protein comprising a bacterial ADP-ribosylating exotoxin fused to a STa exotoxin via a peptide linker for inducing an antigen-specific immune response by epicutaneous administration.

The present invention also provides for a patch and/or formulations for transcutaneous immunizations and/or induction of an immune response comprising a fusion protein, wherein said fusion protein comprises a bacterial ADP-ribosylating exotoxin (bARE) fused to a STa exotoxin. The patch and/or formulation may comprise a fusion protein which further comprises a peptide linker.

In addition, the invention further provides that the bARE is selected from the group consisting of a cholera toxin (CT), heat-labile enterotoxin from E. coli (LT), Pseudomonas exotoxin A (ETA), pertussis toxin (PT), and diphtheria toxin (DT). In another embodiment, the invention provides methods of inducing an immune response in a subject comprising application of different domains of a bacterial ADP ribosylating exotoxin fused to STa, or portions thereof. For example, it is contemplated that the A subunit, or portions thereof, of a bARE can be fused with STa, or portions thereof. In addition, it is contemplated that the B subunit, or portions thereof, of any bARE can be fused with STa. In a specific embodiment, the A subunit of bARE is selected from the group consisting of a cholera toxin A subunit (CTA), heat-labile enterotoxin A subunit from E. coli (LTA), Pseudomonas exotoxin A-A subunit (ETAA), pertussis toxin A subunit (PTA), and diphtheria toxin A subunit (DTA). In another embodiment, the B subunit of bARE is selected from the group consisting of cholera toxin B subunit (CTB), heat-labile enterotoxin B subunit from E. coli (LTB), Pseudomonas exotoxin A-B subunit (ETAB). In an even further embodiment the bARE holotoxin is selected is selected from the group consisting of a cholera toxin (CT-holo), heat-labile enterotoxin from E. coli (LT-holo), Pseudomonas exotoxin A (ETA-holo), pertussis toxin (PT-holo), and diphtheria toxin (DT-holo). It is also it is contemplated that the whole bARE toxin (holotoxin) is fused with the STa. The invention also provides for a patch and/or formulations in which the STa exotoxin is a pro-STa exotoxin.

In specific embodiments, the invention provides that the bARE is LT, or portions thereof, is fused to STa (LT-STa). In another embodiment, the LTB subunit (LTB), or portions thereof, is fused to STa (LTB-STa). In another embodiment, the LTA subunit (LTA) is fused to STa (LTA-STa). In another embodiment the LT, LTA or LTB, or portions thereof, will be fused to the proSTa to create LT-proSTa, LTA-proSTa, or LTB-proSTa fusion proteins, respectively. It is also contemplated that the above identified fusion proteins comprise a peptide linker.

The present invention also provides for methods of inducing an antigen-specific immune response in a subject comprising applying a patch and/or formulations of any of the fusion proteins of the invention to a subject to induce an antigen-specific immune response.

The present invention also provides for methods of preventing a disease in a subject comprising applying a patch and/or a formulation comprising a fusion protein of the invention to a subject to induce an antigen-specific immune response, thereby preventing a disease. In one embodiment the disease is Traveler's diarrhea.

The present invention also provides for methods of treating, preventing, or inhibiting an enterotoxigenic Escherichia coli (ETEC) infection in a subject comprising applying to an area of the skin of said subject a therapeutically effective amount of a formulation comprising a fusion protein containing a bARE fused to a STa exotoxin, thereby inducing an antigen-specific immune response to treat, prevent, or inhibit an ETEC infection.

The transcutaneous immunization system of the present invention can deliver antigen to the immune system through the stratum corneum without physical or chemical penetration to the dermis layer of the skin. This delivery system induces an antigen-specific immune response. Although perforation of intact skin is not required, superficial penetration or micropenetration of the skin can act as an enhancer. Similarly, hydration may enhance the immune response. This system can induce antigen-specific immune effectors after epicutaneous application of a formulation containing one or more active ingredients (e.g., antigen, polynucleotide encoding antigen).

The formulation may initiate and/or enhance processes such as antigen uptake, processing, and presentation; Langerhans cell activation, migration from the skin to other immune organs, and differentiation to mature dendritic cells; contacting antigen with lymphocytes bearing cognate antigen receptors on the cell surface and their stimulation; and combinations thereof.

Other Traveler's diseases of interest that can be treated include campylobacteriosis (Campylobacter jejum), giardiasis (Giardia intestinalis), hepatitis (hepatitis virus A or B), malaria (Plasmodium falciparum, P. vivax, P. ovale, and P. malariae), shigellosis (Shigella boydii, S. dysenteriae, S. flexneri, and S. sonnei), viral gastroenteritis (rotavirus), and combinations thereof. Effectiveness may be assessed by clinical or laboratory criteria.

### BRIEF DESCRIPTION OF THE DRAWINGS

FIG. 1. Construction of LT/pro-STa and LTB/pro-STa fusion. (A) The LT gene (1148 bp), LT-B (378 bp) and the human STa gene (159 bp) were amplified from genomic DNA of the ETEC H10407 strain using the 5′ and 3′ primers listed in Table 1. (B) SDS-PAGE of LT-proSTa and LTB-STa.

FIG. 2. Transcutaneous immunization with LT-STa alone or LT-STa adjuvanted with LT holotoxin. Mice were anesthetized and the dorsal caudal surface at the base of the tail was shaved prior to patch application. The shaved skin was hydrated with saline and pretreated with emery paper to disrupt the stratum corneum. A gauze pad on an adhesive backing was loaded (25 μl) with 25 ug LT-STa fusion protein alone or mixed with 10 ug LT. Patches were applied for 18 hr. All mice were immunized on day 0 and 14 and serum was collected two weeks after the second immunization. An ELISA method was used to detect serum antibodies to STa. FIG. 2 represents titration curves for individual animals.

FIG. 3. Transcutaneous immunization with LT-STa alone or LT-STa adjuvanted with LT holotoxin elicits fecal antibodies to STa. Mice were prepared for immunization as described in FIG. 2. All mice were immunized with three doses of LT-STa alone (25 μg) or mixed with LT holotoxin (10 μg) on day 0, 14 and 28. Fresh fecal samples were collected from individual animals seven days after the third immunization. The samples were homogenized and the clarified extract was assayed for STa-specific IgA (panels A and B) and STa-specific IgG (panels C and D) using an ELISA method. FIGS. A-D represent titration curves for individual animals.

FIG. 4. Construction of prepro-STa and pro-STa fused to the LTA with intervening spacer sequences. This figure shows the 9 mer (gly-ser-glu-phe-glu-leu-arg-arg-pro) (SEQ ID NO: 7) and 4 mer (gly-ser-gly-thr) (SEQ ID NO: 9) spacer sequences placed between the Prepro-STa and pro-STa and fused to the N-terminus of LTA.

FIG. 5. PCR amplification of LT, prepro-STa and pro-STa from ETEC strain H10407.

FIG. 6. Restriction digests of plasmids of pENTR/D containing the LT, prepro-STa and pro-STa gene inserts.

FIG. 7. PCR method was used to demonstrate the proSTa fused to the LTA gene.

FIG. 8. Detection of Prepro-STa/LT and ProSTa/LT Fusion Proteins by Western Blot Analysis.

### DESCRIPTION OF PREFERRED EMBODIMENTS

### Definitions

“Bacterial ADP-ribosylating exotoxins” (referred to as bAREs) represent one family of virulence factors that exert their toxic effects by transferring the ADP-ribose moiety of NAD onto specific eukaryotic target proteins. For example, some bAREs regulate signal transduction, like the heterotrimeric GTP-binding proteins and the low-molecular-weight GTP-binding proteins. Many protein toxins, notably those that act intracellularly (with regard to host cells), consist of two components: one component (subunit A) is responsible for the enzymatic activity of the toxin; the other component (subunit B) is concerned with binding to a specific receptor on the host cell membrane and transferring the enzyme across the membrane. The enzymatic component is not active until it is released from the native (A+B) toxin. Isolated A subunits are enzymatically active but lack binding and cell entry capability. Isolated B subunits may bind to target cells (and even block the binding of the native toxin), but they are nontoxic. It is contemplated that different portions of the bAREs can be can be fused to different antigens like STa. For example, the A subunit, or portions thereof, is fused with STa. The A subunit may or may not be enzymatically active but should be antigenic and/or immunogenic. In addition, the B subunit, or portions thereof, can be fused with STa. The B subunit may or may not have binding activity but should be antigenic and/or immunogenic. It is also it is contemplated that the whole toxin (holotoxin) is fused with the STa. All three constructs can be used together or separately for transcutaneous immunizations.

Heat stable enterotoxin (STa) falls into two classes. The 18 amino acid STa designated STp and the 19 amino acid STa designated STh originated from porcine and human strains, respectively. Both STp and STh are typical extracellular toxins and are synthesized in a Pro-STa form comprising 72 amino acid residues. This indicates that that the STs undergo extensive processing. It is contemplated that all forms of STs are included in the invention. For example, it is contemplated that the LT-STa fusion could comprise the 18 amino acid, the 19 amino acid or the 72 amino acid forms of STs, or variants (including splice, cleaved, or mutated variants) thereof.

“Fusion protein(s) of the invention,” “bacterial ADP ribosylating-STa fusion protein(s)” or “bARE-STa fusion protein(s)” as used herein refer to proteins formed by the fusion of at least one molecule of STa (or a fragment or variant thereof) to at least one molecule of bARE toxin (or fragment or variant thereof). A bARE-STa fusion protein of the invention comprises at least a fragment or variant of a bARE and at least a fragment or variant of STa, which are associated with one another by genetic fusion (i.e., the fusion protein is generated by translation of a nucleic acid in which a polynucleotide encoding all or a portions of the ADP ribosylating exotoxin is joined in-frame with a polynucleotide encoding all or a portion of STa). It is also contemplated that all forms of STa are included in the invention. It is also contemplated that different portions of the bacterial ADP ribosylating exotoxin can be fused to STa. For example, it is contemplated that the A subunit, or portions thereof, of any bARE can be fused with STa. In addition, it is contemplated that the B subunit, or portions thereof, of any bARE can be fused with STa. In one specific embodiment, the bARE is LT, or portions thereof, is fused to Sta (LT-STa). In another embodiment, the LTB subunit (LTB), or portions thereof, is fused to STa (LTB-STa). In another embodiment, the LTA subunit (LTA) is fused to STa (LTA-STa). In another embodiment the LT, LTA or LTB, or portions thereof, will be fused to the proSTa to create LT-proSTa, LTA-proSTa, or LTB-proSTa fusion proteins, respectively. For example, heat stable enterotoxins include the ST enterotoxins from UniProtKB/Swiss Prot accession numbers P01559, Q47185 and P07965. Further, the heat labile enterotoxins (LT) used in the practice of the invention, include, without limitation, the LT subunits and proteins having UniProtKb/SwissProt accession numbers P13810, P13812, P43528, P43529, P43530, P13811, P06717 and P32890. The nucleotide sequences and amino acid sequences of the proteins and subunits having accession numbers above are incorporated by reference herein in their entirety. Use of any one of the B subunits with any one of the A subunits is an option in the practice of the methods and products disclosed in the invention herein. Use of any one of the A, B or both A and B subunits with any one the STa proteins is also an option in the practice of the method and products disclosed in the invention herein. It is also contemplated that the above identified fusion proteins comprise a peptide linker.

A “patch” refers to a product which includes a solid substrate (e.g., occlusive or nonocclusive surgical dressing) as well as at least one active ingredient. Liquid or semi-liquid formulations may be incorporated in a patch. Here, the patch comprises a backing layer, a pressure-sensitive adhesive layer and an immunogenic formulation. The solid substrate is at least the backing layer, but the adhesive and immunogenic formulations may also form part of the solid substrate if they are suitably dried and cured. One or more active components of the immunogenic formulation may be applied on the adhesive layer, incorporated in the adhesive layer, or combinations thereof. Layers may be formed, and then adhered or laminated together.

An “antigen” is an active component of the formulation which is specifically recognized by the immune system of a human or animal subject after immunization or vaccination. It may also be a component in an antigenic composition or formulation. The antigen may comprise a single or multiple immunogenic epitopes recognized by a B-cell receptor (i.e., secreted or membrane-bound antibody) or a T-cell receptor. Proteinaceous epitopes recognized by T-cell receptors have typical lengths and conserved amino acid residues depending on whether they are bound by major histocompatibility complex (MHC) Class I or Class II molecules on the antigen presenting cell. In contrast, proteinaceous epitopes recognized by antibodies may be of variable length including short, extended oligopeptides and longer, folded polypeptides. Single amino acid differences between epitopes may be distinguished. The antigen may be capable of inducing an immune response against a molecule of a pathogen, allergenic substances, or mammalian host (e.g., autoantigens, cancer antigens, molecules of the immune system). For immunoregulation, that molecule may be an allergen, autoantigen, internal image thereof, or other components of the immune system (e.g., B- or T-cell receptor, co-receptor or ligand thereof, soluble mediator or receptor thereof). Thus, antigen is usually identical or at least derived from the chemical structure of the molecule, but mimetics which are only distantly related to such chemical structures may also be successfully used.

An “adjuvant” is an active component of the formulation which assists in inducing an immune response to the antigen. Adjuvant activity is the ability to increase the immune response to a heterologous antigen (i.e., antigen which is a separate chemical structure from the adjuvant) by inclusion of the adjuvant itself in a formulation or in combination with other components of the formulation or particular immunization techniques. As noted above, a molecule may contain both antigen and adjuvant activities by chemically conjugating antigen and adjuvant or genetically (recombinantly) fusing coding regions, or portions thereof, of antigen and adjuvant; thus, the formulation may contain only one ingredient or component. Some naturally-occurring proteins such as CT and LT have both adjuvant and antigenic properties; some recombinant proteins are known to have similar properties (LeIF); some non-protein adjuvants may also induce antibodies to themselves, such as LPS or lipid A. The combination of adjuvant and antigenic qualities may be used to induce protective immune responses. For example, LT antibodies are protective against ETEC, LeIF immune responses are effective in manifestations of Leishmaniasis and LPS antibodies may be protective against diseases caused by gram-negative organisms.

The term “effective amount” is meant to describe that amount of adjuvant or antigen which induces an antigen-specific immune response. A “subunit” immunogen or vaccine is a formulation comprised of active components (e.g., adjuvant, antigen) which have been isolated from other cellular or viral components of the pathogen (e.g., membrane or polysaccharide components like exotoxin) by recombinant techniques, chemical synthesis, or at least partial purification from a natural source.

The term “therapeutically effective amount” as used in the invention, is meant to describe that amount of antigen which induces an antigen-specific immune response. Such induction of an immune response may provide a treatment such as, for example, immunoprotection, desensitization, immunosuppression, modulation of autoimmune disease, potentiation of cancer immunosurveillance, or therapeutic vaccination against an established infectious disease. The amount used will ultimately be determined at the discretion of a physician or veterinarian to achieve a beneficial effect in the treated subject. For example, diseases or other pathologic conditions may be prevented or cured. It is sufficient, however, for the beneficial effect to be a reduction in the number or severity of symptoms associated with the disease or other pathologic condition. Such effects may be measured through objective criteria by the physician or veterinarian, or subjective self-reporting by the subject or observers familiar with the subject.

Perforation of the skin means to cut or pass through with a sharp instrument. For example, using a needle to deliver a medication.

### DETAILED DESCRIPTION

### Transcutaneous Immunization

A system for delivery of an antigen transcutaneously and/or transcutaneous immunization (TCI) is provided which induces an immune response (e.g., humoral and/or cellular effector specific for an antigen) in a human or animal. The delivery system provides simple, epicutaneous application of a formulation comprised of at least one fusion protein of the invention to the skin of a human or animal subject (Glenn et al. (1998) J. Immunol. 161, 3211-3214; Glenn et al. (1998) Nature, 391, 851; Glenn et al. (2000) Nature Med. 6, 1403-1406; Hammond et al. (2000) Adv. Drug Deliv. Rev. 43, 45-55; Scharton-Kersten et al. (2000) Infect. Immun. 68, 5306-5313). An immune response to a fusion protein of the invention is thereby induced with or without chemical and/or physical penetration enhancement and the skin may or may not be perforated through the dermal layer. This delivery system may also be used in conjunction with enteral, mucosal or other parenteral immunization techniques. Thus, the patch technologies described herein could be used for treatment of humans and animals in, for example, immunotherapy and immunoprotection: therapeutically to treat existing disease, protectively to prevent disease, to reduce the severity and/or duration of disease, to ameliorate one or more symptoms of disease, or combinations thereof.

The transit pathways utilized by antigens to traverse the stratum corneum are unknown at this time. The stratum corneum (SC) is the principal barrier to delivery of drugs and antigens through the skin. Transdermal drug delivery of polar drugs is widely held to occur through aqueous intercellular channels formed between the keratinocytes (Transdermal and Topical Drug Delivery Systems, Eds. Ghosh et al., Buffalo Grove: Interpharm Press, 1997). Although the SC is the limiting barrier for penetration, it is breached by hair follicles and sweat ducts. Whether antigens penetrate directly through the SC or via the epidermal appendages may depend on a host of factors. These appendages are thought to play only a minor role in transdermal drug delivery (Barry et al. (1987) J. Control Rel. 6, 85-97). Despite some evidence in mice that transcutaneous immunization using DNA may utilize hair follicles as the pathway for skin penetration (Fan et al. (1999) Nature Biotechnol. 17, 870-872), it is more likely that the robust immune responses utilize more of the skin surface area. Because disruption of the SC barrier can be accomplished by simple hydration of the skin (Pharmaceutical Skin Penetration Enhancement, Eds. Walters et al., New York: Marcel Dekker, 1993), this has been employed for transcutaneous immunization.

Activation of one or more of adjuvant, antigen, and antigen presenting cell (APC) may promote the induction of the immune response. The APC processes the antigen and then presents one or more epitopes to a lymphocyte. Activation may promote contact between the formulation and the APC (e.g., Langerhans cells, other dendritic cells, macrophages, B lymphocytes), uptake of the formulation by the APC, processing of antigen and/or presentation of epitopes by the APC, migration and/or differentiation of the APC, interaction between the APC and the lymphocyte, or combinations thereof. The adjuvant by itself may activate the APC. For example, a chemokine may recruit and/or activate antigen presenting cells to a site. In particular, the antigen presenting cell may migrate from the skin to the lymph nodes, and then present antigen to a lymphocyte, thereby inducing an antigen-specific immune response. Furthermore, the formulation may directly contact a lymphocyte which recognizes antigen, thereby inducing an antigen-specific immune response.

In addition to eliciting immune reactions leading to activation and/or expansion of antigen-specific B-cell and/or T-cell populations, including antibodies and cytotoxic T lymphocytes (CTL), the invention may positively and/or negatively regulate one or more components of the immune system by using transcutaneous immunization to affect antigen-specific helper (Th1 and/or Th2) or delayed-type hypersensitivity T-cell subsets (T_{DTH}). The desired immune response induced is preferably systemic or regional (e.g., mucosal) but it is usually not undesirable immune responses (e.g., atopy, dermatitis, eczema, psoriasis, and other allergic or hypersensitivity reactions). As seen herein, the immune responses induced are of the quantity and quality that provide therapeutic or prophylactic immune responses useful for treating disease.

Hydration of the intact or penetrated skin before, during, or immediately after epicutaneous application of the formulation is preferred and may be required in some or many instances. For example, hydration may increase the water content of the topmost layer of skin (e.g., stratum corneum or superficial epidermis layer exposed by penetration enhancement techniques) above 25%, 50% or 75%. Skin may be hydrated with an aqueous solution of 10% glycerol, 70% isopropyl alcohol, and 20% water. Addition of an occlusive dressing or use of a semi-liquid formulation (e.g., cream, emulsion, gel, lotion, paste) can increase hydration of the skin. For example, lipid vesicles or sugars can be added to a formulation to thicken a solution or suspension. Hydration occurs with or without disruption of all or at least a portion of the stratum corneum at the site of application of the formulation, along with possibly also a portion of the epidermis, as long as the dermis is not perforated. The intent is for the formulation to act on skin antigen presenting cells instead of introducing immunologically-active components of the formulation into the systemic circulation, although some portion of the formulation may act at distal sites.

Skin may be swabbed with an applicator (e.g., adsorbent material on a pad or stick) containing hydration or chemical penetration agents or the agents may be applied directly to skin. For example, aqueous solutions (e.g., water, saline, other buffers), acetone, alcohols (e.g., isopropyl alcohol), detergents (e.g., sodium dodecyl sulfate), depilatory or keratinolytic agents (e.g., calcium hydroxide, salicylic acid, ureas), humectants (e.g., glycerol, other glycols), polymers (e.g., polyethylene or propylene glycol, polyvinyl pyrrolidone), or combinations thereof may be used or incorporated in the formulation. Similarly, abrading the skin (e.g., abrasives like an emery board or paper, sand paper, fibrous pad, pumice), removing a superficial layer of skin (e.g., peeling or stripping with an adhesive tape), microporating the skin using an energy source (e.g., heat, light, sound, electrical, magnetic) or a barrier disruption device (e.g., blade, needle, projectile, spray, tine), or combinations thereof may act as a physical penetration enhancer. See WO 98/29134, WO 01/34185, and WO 02/07813; U.S. Pat. Nos. 5,445,611, 6,090,790, 6,142,939, 6,168,587, 6,312,612, 6,322,808 and 6,334,856 for description of microblades or microneedles, gun or spray injectors, and for microporation of the skin and techniques that might be adapted for transcutaneous immunization. The objective of chemical or physical penetration enhancement in conjunction with TCI is to remove at least the stratum corneum, or a superficial or deeper epidermal layer, without perforating skin through or past the dermal layer. This is preferably accomplished with minor discomfort at most to the human or animal subject and without bleeding at the site. For example, applying the formulation to intact skin may or may not involve thermal, optical, sonic or electromagnetic energy to perforate layers of the skin to below the stratum corneum or epidermis.

The term “penetration enhancer” as used herein refers to those chemicals which when applied in the formulation, before application, during application, or after application results in a disruption of the skin as describe above. Some chemicals (e.g., alcohols) may or may not disrupt the stratum corneum depending on how vigorously they are applied (e.g., swabbing or scrubbing with sufficient pressure). For example, including alcohol, oil-in-water (O/W) or water-in-oil (W/O) emulsions, lipid micelles, or lipid vesicles in the formulation may enhance penetration of one or more immunologically-active ingredients of the same formulation across intact skin without detectable disruption of the stratum corneum.

### Bacterial ADP Ribosylating Exotoxins

Bacterial ADP-ribosylating exotoxins (referred to as bAREs) represent one family of virulence factors that exert their toxic effects by transferring the ADP-ribose moiety of NAD onto specific eukaryotic target proteins. They are usually organized as A:B toxins. Many protein toxins, notably those that act intracellularly (with regard to host cells), consist of two components: one component (subunit A) is responsible for the enzymatic activity of the toxin; the other component (subunit B) is concerned with binding to a specific receptor on the host cell membrane and transferring the enzyme across the membrane. The enzymatic component is not active until it is released from the native (A+B) toxin. Isolated A subunits are enzymatically active but lack binding and cell entry capability. Isolated B subunits may bind to target cells (and even block the binding of the native toxin), but they are nontoxic. There are a variety of ways that toxin subunits may be synthesized and arranged: A+B indicates that the toxin is synthesized and secreted as two separate protein subunits that interact at the target cell surface; A−B or A−5B indicates that the A and B subunits are synthesized separately, but associated by noncovalent bonds during secretion and binding to their target; 5B indicates that the binding domain of the protein is composed of 5 identical B subunits. A/B denotes a toxin synthesized as a single polypeptide, divided into A and B domains that may be separated by proteolytic cleavage. A-B toxins include, but are not limited to, diphtheria, Pseudomonas exotoxin A, cholera toxin (CT), E. coli heat-labile enterotoxin (LT), pertussis toxin, C. botulinum toxin C2, C. botulinum toxin C3, C. limosum exoenzyme, B. cereus exoenzyme, Pseudomonas exotoxin S, Staphylococcus aureus EDIN, and B. sphaericus. It is contemplated that different portions of the bAREs are used as part of the invention. For example, it is contemplated that the A subunit, or portions thereof, can be fused with STa, and that the B subunit, or portions thereof, can be fused with STa, and the whole toxin (holotoxin) can be fused with the STa.

Point mutations (e.g., single, double, or triple amino acid substitutions), deletions (e.g., protease recognition site), and isolated functional domains of bacterial ADP-ribosylating exotoxin are also part of the invention. Derivatives which are less toxic or have lost their ADP-ribosylation activity, but retain their adjuvant activity and antigenic activity have been described. Specific mutants of E. coli heat-labile enterotoxin include LT-K63, LT-R72, LT(H44A), LT(R192G), LT(R192G/L211A), and LT(Δ192-194). The enzymatic activity of a bARE, or portions thereof, may be determined by ADP ribosylating assay and/or receptor binding assays. Toxicity may be assayed with the Y-1 adrenal cell assay (Clements et al. (1979) Infect. Immun. 24, 760-769). The enzymatic activity of a bARE, or portions thereof, may be determined by ADP ribosylating assays and/or receptor binding assays. ADP-ribosylation may be assayed with the NAD-agmatine ADP-ribosyltransferase assay (Moss et al. (1993) J. Biol. Chem. 268, 6383-6387). Particular ADP-ribosylating exotoxins, derivatives thereof, and processes for their production and characterization are described in U.S. Pat. Nos. 4,666,837; 4,935,364; 5,308,835; 5,785,971; 6,019,982; 6,033,673; and 6,149,919. The antigenic and adjuvant functional activity of a bARE, or portions thereof, may be determined in vivo or in vitro assays generally known to those skilled in the art.

Heat Stable E. coli Toxin

Heat stable enterotoxin (STa) is produced by enterotoxigenic strains of E. coli. STa is a major cause of diarrheal diseases in infants in the developing world and in travelers worldwide. STa exerts its toxic effects at the level of the mammalian small intestine where it causes fluid accumulation by specific binding to the high-affinity transmembrane guanylate cyclase C receptor present on the intestinal enterocytes (Schulz et al. (1990) Cell 63, 941-948). STa falls into two classes. The 18 amino acid STa designated STp and the 19 amino acid STa designated STh originated from porcine and human strains, respectively. Both STp and STh are typical extracellular toxins and are synthesized in a Pre-Pro-STa form comprising 72 amino acids residues. The Pre region functions as a leader peptide, the pro-region is cleaved in the periplasmic space. The invention also contemplates point mutations of STs. Point mutations include mutations that reduce toxicity, and/or increase antigenicity, and/or reduce or remove splice variants (including all naturally occurring variants and/or alleles).

Because of its small size (19 amino acids), Stable Toxin (ST) is poorly immunogenic. However, the immunogenicity of ST is improved as a hapten to a carrier molecule such as albumin. The present invention provides an antitoxin vaccine comprising genetic fusions of LT(holotoxin)-STa, LTA-STa, and LTB-STa fusion proteins have been engineered and demonstrated to improve the immunogenicity of ST. The present invention also provides fusions proteins comprising portions of the A subunit and/or B subunit of LT fused to STa.

### Fusion Proteins

STa is poorly immunogenic. For this reason a number of efforts have been made to develop genetic fusions between STa and several proteins to elicit neutralizing and protective antibodies raised against the native STa structure. These fusion proteins include fusions with E. coli heat-labile enterotoxin, cholera toxin and others. These earlier studies have shown that LT-proSTa fusion proteins are immunogenic and do result in the generation of toxin neutralizing antibodies to STa and to LT. However, the immunization methods were with repeated doses administered by intraperitoneal injection and/or peroral route. Although these investigations demonstrate the increased immunity to STa, the route of immunization is not practical for a human vaccine. In addition, in most cases, no hybrid protein with properly folded STa joined covalently to the carrier protein was both extracellularly secreted and fully active (Batisson et al (2000) Infection and Immunity 68, 4064-4074). Although a LT-STa fusion is contemplated for transcutaneous immunizations, an alternative strategy comprises a seven amino acid linker between LT and STa, and/or LTA and STa and/or LTB and STa. LT-STa fusion protein was immunogenic in that antibodies produced against STa were capable of neutralizing native STa.

“Bacterial ADP ribosylating-STa fusion” proteins or “bARE-STa” refer to a protein formed by the fusion of at least one molecule of STa, or a fragment, portion or variant thereof, wherein said STa fragment, portion or variant thereof retains its functional activity and/or its immunogenic and/or antigenic activity, to at least one molecule of bARE toxin, or fragment, portion or variant thereof, wherein said bARE molecule fragment, portion or variant thereof retains its functional activity and/or its immunogenic and/or antigenic activity. A bARE-STa fusion protein of the invention comprises at least a fragment or variant of a bARE and at least a fragment or variant of STa, which are associated with one another by genetic fusion (i.e., the fusion protein is generated by translation of a nucleic acid in which a polynucleotide encoding all or a portions of the ADP ribosylating exotoxin is joined in-frame with a polynucleotide encoding all or a portion of STa). It is also contemplated that all forms of STs are included in the invention. For example it is contemplated that the LT-STa fusion could be the 18 amino acid, the 19 amino acid, or the 72 amino acid forms of STs, or variants thereof (including spliced, cleaved and/or mutated variants). The invention also contemplates point mutations of STs. Point mutations include mutations that reduce toxicity, and/or increase antigenicity, and/or reduce or remove splice variants (including all naturally occurring variants and/or alleles). All variants mentioned above will be identified as “STa”. The bacterial ADP ribosylating exotoxin fusion protein of the invention includes, but is not limited to, a toxin selected from the group consisting of cholera holotoxin (CT), heat-labile holotoxin from E. coli (LT), Pseudomonas A holotoxin (ETA), pertussis holotoxin (PT) and diphtheria holotoxin (DT) fused to Sta.

Many bAREs consist of two components: one component (subunit A) is responsible for the enzymatic activity of the toxin; the other component (subunit B) is concerned with binding to a specific receptor on the host cell membrane and transferring the enzyme across the membrane. Thus, the present invention provides different portions of the bacterial ADP ribosylating exotoxin fused to STa. For example, the A subunit, or portions thereof, of any bARE can be fused with STa, or portions thereof. In addition, the present invention provides the B subunit, or portions thereof, of any bARE fused with STa, or portions thereof. In one specific embodiment, the bARE is LT, or portions thereof, fused to Sta (LT-STa). In another embodiment, the LTB subunit (LTB), or portions thereof, is fused to STa (LTB-STa). In another embodiment, the LTA subunit (LTA) is fused to STa (LTA-STa). In another embodiment the LT, LTA or LTB, or portions thereof, will be fused to the proSTa to create LT-proSTa, LTA-proSTa, or LTB-proSTa fusion proteins, respectively. It is also contemplated that any of the above constructs can be used together or separately for transcutaneous immunizations.

Additionally, the ADP-ribosylating-STa fusion protein of the invention may include a peptide linker between the fused portions to provide greater physical separation between the moieties and thus maximize the ability for each domain to fold into its natural conformation without hindrance from other domains. The linker peptide may consist of amino acids such that it is flexible or more rigid. The fusion protein may include an amino acid linker of approximately 200, 150, 100, 50, 40, 30, 20, 10, 9, 8, 7, 6, 5, 4, 3, or 2 residues. Preferably, the linker comprises SEQ ID NO: 7 or SEQ ID NO: 9. In a one embodiment, the invention comprises a fusion protein comprising a bacterial ADP-ribosylating exotoxin (bARE) fused to a STa exotoxin, fused via a peptide linker. In other embodiments LT-STa, LTA-STa, LTB-STa, and LT-holo-STa are fused via a peptide linker having SEQ ID NO: 7 or SEQ ID NO: 9. More preferably, the peptide linker consists of the amino acid sequence as set forth in SEQ ID NO: 7 or SEQ ID NO: 9.

There have been several publications describing chemical conjugations between LT and STa (see, Klipstein et al. (1982) Infect. Immun. 37, 550-557; U.S. Pat. No. 4,411,888; WO 02/064162). Chemical conjugation, however, is extremely cumbersome, time consuming and unpredictable. There are many steps that must be completed in order to have the desired product. Initially, the proteins must be purified separately and quantified. Next, the purified proteins and the chemical conjugate must be added into a reaction mixture under the correct stoichiometry between each protein and the chemical conjugate, in an optimal temperature, for a precise amount of time. After the reaction has occurred, another purification step is required to remove the chemical linker and any unconjugated proteins. In some cases, the proteins have to be modified in order to conjugate them. This modification can cause the protein to loose its natural conformation and, therefore, may not be effective as a vaccine. In other cases, the chemical conjugate itself may be cleaved in vitro over time, thus reducing shelf life, or be cleaved in vivo, thus reducing potency. The chemical conjugate also may create antigenicity reactions which could lead to reduced potency or insurmountable regulatory hurtles. Since chemical conjugation is extremely cumbersome and time consuming, it may even prevent efficient commercial production of a vaccine. In addition, due to regulatory requirements, the ratio of the conjugated proteins may have to be established. This may require additional release tests which may be expensive and time consuming. Because fusion proteins are a single polypeptide it usually requires one purification step, thus, saving time and money. Therefore, the inventors believe that using fusions proteins are cheaper and a more predicable way to manufacture vaccines, thus making a fusion protein vaccine more commercially viable.

Point mutations (e.g., single, double, or triple amino acid substitutions), deletions (e.g., protease recognition site), and isolated functional portions of bacterial ADP-ribosylating exotoxin are also contemplated as part of the invention for the use in bARE-STa fusion proteins. Derivatives which are less toxic or have lost their ADP-ribosylation activity, but retain their adjuvant activity have been described and may be fused to STa. Specific mutants of E. coli heat-labile enterotoxin include LT-K63, LT-R72, LT(H44A), LT(R192G), LT(R192G/L211A), and LT(A192-194). Toxicity may be assayed with the Y-1 adrenal cell assay (Clements et al. (1979) Infect. Immun. 24, 760-769). ADP-ribosylation may be assayed with the NAD-agmatine ADP-ribosyltransferase assay (Moss et al. (1993) J. Biol. Chem. 268, 6383-6387). Particular ADP-ribosylating exotoxins, derivatives thereof, and processes for their production and characterization are described in U.S. Pat. Nos. 4,666,837; 4,935,364; 5,308,835; 5,785,971; 6,019,982; 6,033,673; and 6,149,919.

The present invention provides nucleic acid molecules encoding bARE-STa fusion proteins comprising a bARE toxin, or a portion thereof, fused to STa, or portion thereof, for transcutaneous immunizations. The fusion protein may further comprise a linker region, for instance a linker of approximately 200, 150, 100, 50, 40, 30, 20, 10, 9, 8, 7, 6, 5, 4, 3, or 2 amino acid residues. Nucleic acid molecules of the invention may be purified or not.

Host cells and vectors for replicating the nucleic acid molecules and for expressing the encoded fusion proteins are also provided. Any vectors or host cells may be used, whether prokaryotic or eukaryotic, but prokaryotic expression systems, in particular E. coli expression systems, may be preferred. Many vectors and host cells are known in the art for such purposes. It is well within the skill of the art to select an appropriate set for the desired application.

DNA sequences encoding bAREs, or portions thereof, and STa, or portions thereof, may be cloned from a variety of genomic or cDNA libraries known in the art. The techniques for isolating such DNA sequences using probe-based methods are conventional techniques and are well known to those skilled in the art. Probes for isolating such DNA sequences may be based on published DNA or protein sequences (see, for example, Baldwin (1993) Comp. Biochem. Physiol. 104, 55-61). Alternatively, the polymerase chain reaction (PCR) method disclosed by Mullis et al (U.S. Pat. No. 4,683,195) and Mullis (U.S. Pat. No. 4,683,202), incorporated herein by reference may be used. The choice of library and selection of probes for the isolation of such DNA sequences is within the level of ordinary skill in the art.

The present invention is also directed to nucleic acid molecules which comprise, or alternatively consist of, a nucleotide sequence which is at least 80%, 85%, 90%, 95%, 96%, 97%, 98% or 99% similar to, for example, the nucleotide coding the sequence comprising the fusion proteins of the invention or the complementary strand thereto. Polynucleotides which hybridize to these nucleic acid molecules under stringent hybridization conditions or lower stringency conditions are also encompassed by the invention, as are polypeptides encoded by these polynucleotides.

The present invention is also directed to polypeptides which comprise, or alternatively consist of, an amino acid sequence which is at least 80%, 85%, 90%, 95%, 96%, 97%, 98%, 99 similar to, for example, the polypeptide sequence comprising the fusion proteins of the invention.

As known in the art “similarity” between two polynucleotides or polypeptides is determined by comparing the nucleotide or amino acid sequence and its conserved nucleotide or amino acid substitutes of one polynucleotide or polypeptide to the sequence of a second polynucleotide or polypeptide. Also known in the art is “identity” which means the degree of sequence relatedness between two polypeptide or two polynucleotide sequences as determined by the identity of the match between two strings of such sequences. Both identity and similarity can be readily calculated (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991).

While there exist a number of methods to measure identity and similarity between two polynucleotide or polypeptide sequences, the terms “identity” and “similarity” are well known to skilled artisans (Sequence Analysis in Molecular Biology, von Heinje, G., Academic Press, 1987; Sequence Analysis Primer, Gribskov, M. and Devereux, J., eds., M Stockton Press, New York, 1991; and Carillo, H., and Lipman, D., SIAM J. Applied Math., 48:1073 (1988)).

Methods commonly employed to determine identity or similarity between two sequences include, but are not limited to those disclosed in “Guide to Huge Computers,” Martin J. Bishop, ed., Academic Press, San Diego, 1994, and Carillo, H., and Lipman, D., SIAM J. Applied Math. 48:1073 (1988). Preferred methods to determine identity are designed to give the largest match between the two sequences tested. Methods to determine identity and similarity are codified in computer programs. Preferred computer program methods to determine identity and similarity between two sequences include, but are not limited to, GCG program package (Devereux, et al. (1984) Nucleic Acids Research 12, 387), BLASTP, BLASTN, FASTA (Atschul, et al. (1990) J. Molec. Biol. 215, 403). The degree of similarity or identity referred to above is determined as the degree of identity between the two sequences indicating a derivation of the first sequence from the second. The degree of identity between two nucleic acid sequences may be determined by means of computer programs known in the art such as GAP provided in the GCG program package (Needleman et al. (1970) J. Mol. Biol. 48, 443-453). For purposes of determining the degree of identity between two nucleic acid sequences for the present invention, GAP is used with the following settings: GAP creation penalty of 5.0 and GAP extension penalty of 0.3.

### Vectors

Expression units for use in the present invention will generally comprise the following elements, operably linked in a 5′ to 3′ orientation: a transcriptional promoter, a secretory signal sequence, a DNA sequence encoding fusion proteins of the invention comprising bAREs, or portions thereof joined to a DNA sequence encoding STa, or portion thereof, and a transcriptional terminator. The selection of suitable promoters, signal sequences and terminators will be determined by the selected host cell and will be evident to one skilled in the art and are discussed more specifically below.

Mammalian expression vectors for use in carrying out the present invention will include a promoter capable of directing the transcription of fusion proteins of the invention, preferably LT-STa fusion protein. Preferred promoters include viral promoters and cellular promoters. Preferred viral promoters include the major late promoter from adenovirus 2 (Kaufman et al. (1982) Mol. Cell. Biol. 2, 1304-13199) and the SV40 promoter (Subramani et al. (1981) Mol. Cell. Biol. 1, 854-864). Preferred cellular promoters include the mouse metallothionein-1 promoter (Palmiter et al. (1983) Science 222, 809-814) and a mouse V6 (see, U.S. Pat. No. 6,291,212) promoter (Grant et al. (1987) Nuc. Acids Res. 15, 5496). A particularly preferred promoter is a mouse V_{H} (see, U.S. Pat. No. 6,291,212) promoter. Such expression vectors may also contain a set of RNA splice sites located downstream from the promoter and upstream from the DNA sequence encoding the bARE-STa fusion protein. Preferred RNA splice sites may be obtained from adenovirus and/or immunoglobulin genes.

Also contained in the expression vectors is a polyadenylation signal located downstream of the coding sequence of interest. Polyadenylation signals include the early or late polyadenylation signals from SV40 the polyadenylation signal from the adenovirus 5 E1B region and the human growth hormone gene terminator (DeNoto et al., (1981) Nuc. Acids Res. 9, 3719-3730). A particularly preferred polyadenylation signal is the V_{H} (see, U.S. Pat. No. 6,291,212) gene terminator. The expression vectors may include a noncoding viral leader sequence, such as the adenovirus 2 tripartite leader, located between the promoter and the RNA splice sites. Preferred vectors may also include enhancer sequences, such as the SV40 enhancer (see, U.S. Pat. No. 6,291,212) and the mouse enhancer (Gillies (1983) Cell 33, 717-728). Expression vectors may also include sequences encoding the adenovirus VA RNAs.

### Formulations

The present invention provides compositions and formulations for transcutaneous induction of an immune response and/or immunizations. Formulations which are useful for inducing an immune response (antigenic compositions and/or formulations) and/or vaccinations of a mammal are provided as well as processes for their manufacture. See related U.S. Pat. Nos. 5,910,306 and 6,797,276; and U.S. application Ser. Nos. 10/472,589, 10/790,715, 09/266,803, which are incorporated herein by reference in their entireties.

The fusion proteins of the invention used for transcutaneous immunization systems may be applied directly on the skin and allowed to air dry; rubbed into the skin or scalp (i.e., massaging); placed on the ear, inguinal, or intertriginous regions, especially for animals with skin that is not readily accessible or to limit self-grooming; held in place with a dressing, patch, or absorbent material; applied by bathing an exposed skin surface or immersing a body part; otherwise held in place by a device such as a stocking, slipper, glove, or shirt; or sprayed onto the skin to maximize contact with the skin. The formulation may be applied in an absorbent dressing or gauze. The formulation may be covered with an occlusive dressing such as, for example, AQUAPHOR (an emulsion of petrolatum, mineral oil, mineral wax, wool wax, panthenol, bisabol, and glycerin from Beiersdorf), COMFEEL (Coloplast), plastic film, or vaseline; or a non-occlusive dressing such as, for example, DUODERM (3M), OPSITE (Smith & Napheu), or TEGADERM (3M). An occlusive dressing excludes the passage of water. The formulation may be applied to single or multiple sites, single or multiple limbs, or large surface areas of the skin by bathing or immersion in a container. The formulation may be applied directly to the skin. One or more components of the formulation may be provided in dry form.

The formulation may be in liquid or semi-liquid form. For example, the formulation may be provided as a liquid: cream, emulsion, gel, lotion, ointment, paste, solution, suspension, or other liquid forms. Formulation may be air dried, dried with elevated temperature, freeze or spray dried, coated or sprayed on a solid substrate and then dried, dusted on a solid substrate, quickly frozen and then slowly dried under vacuum, or combinations thereof to a low moisture content. Adhesive formulations may be cured to a desired amount of crosslinking by suitable choice of initiator, rate accelerator or decelerator, and terminator.

Suitable procedures for making the various dosage forms and production of topical formulations are known. The size of each dose and the interval of dosing to the subject may be used to determine a suitable size and shape of the container, compartment, or chamber. Formulations will contain an effective amount of the active ingredients (e.g., at least one adjuvant and/or one or more antigens) together with carrier or suitable amounts of vehicle in order to provide pharmaceutically-acceptable compositions suitable for administration to a human or animal. Formulations that include a vehicle may be in the form of a cream, emulsion, gel, lotion, ointment, paste, solution, suspension, or other liquid forms known in the art; especially those that enhance skin hydration.

A “patch” refers to a product which includes a solid substrate (e.g., occlusive or nonocclusive surgical dressing) as well as at least one active ingredient. Liquid or semi-liquid formulations may be incorporated in a patch. In one embodiment of the invention, the fusion proteins of the invention are an active ingredient in the patch. In another embodiment the fusion protein comprises a bARE selected from the groups consisting of cholera toxin (CT), heat-labile enterotoxin from E. coli (LT), Pseudomonas exotoxin A (ETA), pertussis toxin (PT), and diphtheria toxin (DT), or portions or fragments thereof. Other ADP-ribosylating toxins are also contemplated as part of the invention.

The present invention provides different portions of the bacterial ADP ribosylating exotoxin fused to STa to be included as part of the active ingredient in a patch formulation. For example, the A subunit, or portions thereof, of any bARE can be fused with STa. In addition, the B subunit, or portions thereof, of any bARE can be fused with STa. Also the whole bARE toxin (holotoxin) is fused with the STa. In one embodiment, the bARE is LT, or portions thereof, fused to Sta (LT-STa). In another embodiment, the LTB subunit (LTB), or portions thereof, is fused to STa (LTB-STa). In another embodiment, the LTA subunit (LTA) is fused to STa (LTA-STa). In an alternate embodiment, the LT, LTA or LTB, or portions thereof, are fused to the proSTa to create LT-proSTa, LTA-proSTa, or LTB-proSTa fusion proteins, respectively. Moreover, the present invention provides the LT holotoxin (LT-holo), or portions thereof, fused to STa (LT-holo-Sta). In another embodiment the present invention provides the LT, LTA or LTB, or portions thereof, and the LT-holo fused to the proSTa to create LT-proSTa, LTA-proSTa, LTB-proSTa, or LT-holo-proSTa fusion proteins, respectively. Preferably the fusion proteins of the invention include a linker peptide between the fused portions to provide greater physical separation between the moieties. In one embodiment the linker is SEQ ID NO: 7 or SEQ ID NO: 9. The invention also provides that all the above constructs can be used together or separately for transcutaneous immunizations.

Suitable procedures for making the various dosage forms and production of patches are known. The size of each dose and the interval of dosing to the subject may be used to determine a suitable size and shape of the container, compartment, or chamber. Formulations will contain an effective amount of the active ingredients (e.g., at least one adjuvant and/or one or more antigens) together with carrier or suitable amounts of vehicle in order to provide pharmaceutically-acceptable compositions suitable for administration to a human or animal. Formulations that include a vehicle may be in the form of a cream, emulsion, gel, lotion, ointment, paste, solution, suspension, or other liquid forms known in the art; especially those that enhance skin hydration. For a patch, successive coatings of formulation may be applied to the substrate or several formulation-containing layers may be laminated to increase its capacity for active ingredients.

Patch material may be nonwoven or woven (e.g., gauze dressing). Layers may also be laminated during processing. It may be nonocclusive or occlusive, but the latter is preferred for backing layers. The optional release liner preferably does not adsorb significant amounts of the formulation, perhaps by treating a film with silicone or fluorocarbon. The patch is preferably hermetically sealed for storage (e.g., foil packaging). The patch can be held onto the skin and components of the patch can be held together using various adhesives. One or more of the adjuvant and/or antigen may be applied to and/or incorporated in the adhesive portion of the patch. Generally, patches are planar and pliable, and they are manufactured with a uniform shape. Optional additives are plasticizers to maintain pliability of the patch, tackifiers to assist in adhesion between patch and skin, and thickeners to increase the viscosity of the formulation at least during processing.

Metal foil, cellulose, cloth (e.g., acetate, cotton, rayon), acrylic polymer, ethylenevinyl acetate copolymer, polyamide (e.g., nylon), polyester (e.g., poly-ethylene naphthalate, ethylene terephthalate), polyolefin (e.g., polyethylene, poly-propylene), polyurethane, polyvinylidene chloride (SARAN), natural or synthetic rubber, silicone elastomer, and combinations thereof are examples of patch materials (e.g., dressing, backing layer, release liner).

The adhesive may be an aqueous-based adhesive (e.g., acrylate or silicone). Acrylic adhesives are available from several commercial sources. Acrylic polymers may be a copolymer of C4-C18 aliphatic alcohol with methacrylic alkyl ester or the copolymer of methacrylic alkyl ester having C4-C18 alkyl, methacrylic acid, and/or other functional monomers. Examples of the methacrylic alkyl ester may include butyl acrylate, isobutyl acrylate, hexyl acrylate, octyl acrylate, 2-ethylhexyl acrylate, iso-octyl acrylate, decyl acrylate, isodecyl acrylate, lauryl acrylate, stearyl acrylate, methyl methacrylate, ethyl methacrylate, butyl methacrylate, isobutyl methacrylate, 2-ethylhexyl methacrylate, iso-octyl methacrylate, decyl methacrylate, etc.

Examples of the functional monomers may include a monomer containing hydroxyl group, a monomer containing carboxyl group, a monomer containing amide group, a monomer containing amino group. The monomer containing hydroxyl group may include hydroxyalkyl methacrylate such as 2-hydroxyethyl methacrylate, hydroxypropyl methacrylate and the like. The monomer containing carboxyl group may include α-β unsaturated carboxylic acid such as acrylic acid, methacrylic acid and the like; maleic mono alkyl ester such as butyl malate and the like; maleic acid; fumaric acid; crotonic acid and the like; and anhydrous maleic acid. Examples of the monomer containing amide group may include alkyl methacrylamide such as acryl-amide, dimethyl acrylamide, diethyl acrylamide and the like; alkylethylmethylol methacrylamide such as butoxymethyl acrylamide, ethoxymethyl acrylamide and the like; diacetone acrylamide; vinyl pyrrolidone; dimethyl aminoacrylate. In addition to the above exemplified monomers for copolymerization, vinyl acetate, styrene, α-methylstyrene, vinyl chloride, acrylonitrile, ethylene, propylene, butadiene and the like may be employed.

Commercially available acrylic adhesives are sold under the tradenames AROSET, DUROTAK, EUDRAGIT, GELVA, and NEOCRYL. EUDRAGIT polymers form a diverse family of polymers whose common feature is a polyacrylic or poly-methacrylic backbone that is compatible with the gastrointestinal tract and which have been widely used in pharmaceutical preparations, especially as coatings for tablets, but it has also been used as a coating for other medical devices. EUDRAGIT polymers are characterized as (1) an anionic copolymer based on methacrylic acid and methylmethacrylate wherein the ratio of free carboxyl groups to the ester groups is approximately 1:1, (2) an anionic copolymer based on methacrylic acid and methylmethacrylate wherein the ratio of free carboxyl groups to the ester groups is approximately 1:2, (3) a copolymer based on acrylic and methacrylic acid esters with a low content of quaternary ammonium groups wherein the molar ratio of the ammonium groups to the remaining neutral methacrylic acid esters is 1:20, and (4) a copolymer based on acrylic and methacrylic acid esters with a low content of quarternary ammonium groups wherein the molar ratio of the ammonium groups to the remaining neutral methacrylic acid esters is 1:40. The copolymers are sold under tradenames EUDRAGIT L, EUDRAGIT S, EUDRAGIT RL, and EUDRAGIT RS. EUDRAGIT E is a cationic copolymer based on diethylaminoethyl methacrylate and neutral methacrylic acid esters; EUDRAGIT NE is a neutral copolymer of polymethacrylates. For methacrylate or acrylate polymers, there are EUDRAGIT RS, EUDRAGIT RL, and EUDRAGIT NE; also available are EUDRAGIT RS-100, EUDRAGIT L-90, EUDRAGIT NE-30, EUDRAGIT L-100, EUDRAGIT S-100, EUDRAGIT E-100, EUDRAGIT RL-100, EUDRAGIT RS-100, EUDRAGIT RS-30D, EUDRAGIT E-100R, and EUDRAGIT RTM.

Furthermore, for the purpose of increasing or decreasing the water absorption capacity of an adhesive layer, the acrylic polymer may be copolymerized with hydrophilic monomer, monomer containing carboxyl group, monomer containing amide group, monomer containing amino group, and the like. Rubbery or silicone resins may be employed as the adhesive resin; they may be incorporated into the adhesive layer with a tackifying agent or other additives.

Alternatively, the water absorption capacity of the adhesive layer can be also regulated by incorporating therein highly water-absorptive polymers, polyols, and water-absorptive inorganic materials. Examples of the highly water-absorptive resins may include mucopolysaccharides such as hyaluronic acid, chondroitin sulfate, dermatan sulfate and the like; polymers having a large number of hydrophilic groups in the molecule such as chitin, chitin derivatives, starch and carboxy-methylcellulose; and highly water-absorptive polymers such as polyacrylic, polyoxyethylene, polyvinyl alcohol, and polyacrylonitrile. Examples of the water-absorptive inorganic materials, which may incorporated into the adhesive layer to regulate its water absorptive capacity, may include powdered silica, zeolite, powdered ceramics, and the like.

The plasticizer may be a trialkyl citrate such as, for example, acetyl-tributyl citrate (ATBC), acetyl-triethyl citrate (ATEC), and triethyl citrate (TEC). The plasticizer may be between 0.001% (w/v) and 5% (w/v) of the adhesive formulation. A suitable concentration may be empirically determined by selecting for pliability of the adhesive layer, and avoiding brittleness.

Exemplary tackifiers are glycols (e.g., glycerol, 1,3 butanediol, propylene glycol, polyethylene glycol); average molecular weights of 200, 300, 400, 800, 3000, etc. are available for the polyakylene glycols. Succinic acid is another tackifier. The tackifier may be between 0.1% (w/w) and 10% (w/w) of the adhesive formulation. A suitable concentration may be empirically determined by avoiding brittleness of the adhesive layer and its pliability.

Thickeners can be added to increase the viscosity of an adhesive or immunogenic formulation. The thickener may be a hydroxyalkyl cellulose or starch, or water-soluble polymers: for example, poloxamers, polyethylene oxides and derivatives thereof, polyethyleneimines, polyethylene glycols, and polyethylene glycol esters. But any molecule which serves to increase the viscosity of a solution may be suitable to improve handling of a formulation during manufacture of a patch. For example, hydroxyethyl or hydroxypropyl cellulose may be between 1% (w/w) and 10% (w/w) of the adhesive or immunogenic formulation. The formulation as a layer may be film cast or extruded, and then layers may be coated or laminated during manufacture of a patch. The capacity for protein might be increased by successive coatings or laminating several thin, adhesive layers together. Alternatively, a viscous formulation may be spread on a substrate (e.g., backing or adhesive layer) with minimal loss of immunologically-active ingredients like adjuvant or antigen. Thickeners are sold as NATROSOL hydroxyethyl cellulose and KLUCEL hydroxypropyl cellulose.

The moisture content of the adhesive layer may be more than 0.5%, more than 1%, more than 2%, less than 10%, less than 5%, less than 2%, and intermediate ranges thereof. The patch may be a pliable, planar substrate from about 1 cm² to about 100 cm². An effective amount of the protein is provided by a single patch. For example, the patch may comprise an amount of protein between 1 μg and 1 mg, 5 μg and 500 μg, 10 μg and 100 μg, or intermediate ranges thereof. Depending on the immunologic activity of the protein, the effective amount of a particular protein may vary. The patch may be stored in a moisture-proof package (e.g., blister pack, foil pouch) for at least one or two years at room temperature (e.g., 20° C. to 30° C.) with an immunological activity between 85% and 115% of the patch's initial activity.

Gel and emulsion systems can be incorporated into patch delivery systems, or be manufactured separately from the patch, or added to the patch prior to application to the human or animal subject. Gels or emulsions may serve the same purpose of facilitating manufacture by providing a viscous formulation that can be easily manipulated with minimal loss. The term “gel” refers to covalently crosslinked, noncrosslinked hydrogel matrices. Hydrogels can be formulated with at least one protein with immunologic activity for PIA patches. Additional excipients may be added to the gel systems that allow for the enhancement of antigen/adjuvant delivery, skin hydration and protein stability. The term “emulsion” refers to formulations such as water-in-oil creams, oil-in-water creams, ointments and lotions. Emulsion systems can be either micelle-based, lipid vesicle-based, or both micelle- and lipid vesicle-based. Emulsion systems can be formulated with at least one adjuvant and/or antigen as the protein-in-adhesive systems. Additional excipients may be added to the emulsion systems that allow for the enhancement of antigen/adjuvant delivery, skin hydration and protein stability.

The formulation may be applied with a patch in contact with skin of the subject. It may be covered with a nonocclusive or occlusive backing layer. The latter prevents evaporation and traps moisture at the site of application. Such a formulation may be applied to single or multiple sites, to single or multiple limbs, or to a large surface area of skin. Other substrates that may be used are pressure-sensitive adhesives such as acrylics, polyisobutylenes, and silicones. The formulation may be incorporated directly into such substrates, perhaps with the adhesive per se instead of adsorption to a porous pad (e.g., cotton gauze) or bilious strip (e.g., cellulose paper).

The adhesive and immunogenic formulations may be at least partially mixed or even thoroughly blended, and then adhered to the backing layer. The immunologically-active ingredient may be dispersed or dissolved in the formulation. Alternatively the immunogenic formulation may be applied to the surface of the adhesive layer by coating or spreading over the adhesive using a Meyer rod, casting a layer and then laminating in close apposition with the adhesive using a roller, printing on the adhesive using a rotogravure, etc. Adhesive may be brought into contact with a release liner. Adhesive and immunogenic formulations may also be brought into contact with microblade or microneedle arrays or tines by coating, dipping the device into the formulation and drying, or spraying the device with the formulation.

Polymers added to the formulation may act as a stabilizer or other excipient of an active ingredient as well as reducing the concentration of the active ingredient that saturates a solution used to hydrate an at least partially-dried form (i.e., dry or semi-liquid) of the active ingredient. Such reduction occurs because the polymer reduces the effective free volume by filling “empty” space in the solvent. In this way, quantities of adjuvant/antigen can be conserved without reducing the amount of saturated solution. An important thermodynamic consideration is that an active ingredient in the saturated solution will be “driven” into regions of lower concentration (e.g., through the skin). For dispersal or dissolution of at least one adjuvant and/or one or more antigens, polymers can also stabilize the adjuvant/antigen-activity of those components of the formulation. Such polymers include ethylene or propylene glycol, vinyl pyrrolidone, and β-cyclodextrin polymers and copolymers.

Formulations in liquid or semi-liquid form may be applied with one or more adjuvants and/or antigens both at the same or separate sites or simultaneously or in frequent, repeated applications. The patch may include a controlled-release reservoir or a rate-controlling matrix or membrane may be used which allows stepped release of adjuvant and/or antigen. It may contain a single reservoir with adjuvant and/or antigen, or multiple reservoirs to separate individual antigens and adjuvants. The patch may include additional antigens such that application of the patch induces an immune response to multiple antigens. In such a case, antigens may or may not be derived from the same source, but they will have different chemical structures so as to induce an immune response specific for different antigens. Multiple patches may be applied simultaneously; a single patch may contain multiple reservoirs. For effective treatment, multiple patches may be applied at intervals or constantly over a period of time; they may be applied at different times, for overlapping periods, or simultaneously.

Solids (e.g., particles of nanometer or micrometer dimensions) may also be incorporated in the formulation. Solid forms (e.g., nanoparticles or microparticles) may aid in dispersion or solubilization of active ingredients; assist in carrying the formulation through superficial layers of the skin; provide a point of attachment for adjuvant, antigen, or both to a substrate that can be opsonized by antigen presenting cells, or combinations thereof. Ingredients that are insoluble or poorly soluble in an aqueous solution may be formulated in an emulsion, lipid vesicles, or micelles.

The invention also encompasses a composition formulated for transcutaneous immunizations containing an effective amount of a fusion protein comprising a bacterial ADP-ribosylating exotoxin fused to a STa exotoxin for inducing an antigen-specific immune response by epicutaneous administration.

A single or unit dose of formulation suitable for administration is provided. The amount of adjuvant or antigen in the unit dose may be anywhere in a broad range from about 0.001 μg to about 10 mg. This range may be from about 0.1 μg to about 1 mg; a narrower range is from about 5 μg to about 500 μg. Other suitable ranges are between about 1 μg and about 10 μg, between about 10 μg and about 50 μg, between about 50 μg and about 200 μg, and between about 1 mg and about 5 mg. A preferred dose for a toxin is about 50 μg or 100 μg or less (e.g., from about 1 μg to about 50 μg or 100 μg). The ratio between antigen and adjuvant may be about 1:1 (e.g., a bacterial ADP-ribosylating exotoxin when it is both antigen and adjuvant) but higher ratios may be suitable for poor antigens (e.g., about 1:10 or less), or lower ratios of antigen to adjuvant may also be used (e.g., about 10:1 or more).

A formulation comprising fusion proteins of the invention or polynucleotide may be applied to skin of a human or animal subject, antigen is presented to immune cells, and an antigen-specific immune response is induced. This may occur before, during, or after infection by pathogen. Only antigen or polynucleotide encoding antigen may be required, but no additional adjuvant, if the immunogenicity of the formulation is sufficient to not require adjuvant activity. The formulation may include an additional antigen such that application of the formulation induces an immune response against multiple antigens (i.e., multivalent). In such a case, antigens may or may not be derived from the same source, but the antigens will have different chemical structures so as to induce immune responses specific for the different antigens. Antigen-specific lymphocytes may participate in the immune response and, in the case of participation by B lymphocytes, antigen-specific antibodies may be part of the immune response. The formulations described above may include binders, buffers, colorings, dessicants, diluents, humectants, preservatives, stabilizers, other excipients, adhesives, plasticizers, tackifiers, thickeners, and patch materials known in the art.

The formulation may be epicutaneously applied to skin to prime or boost the immune response in conjunction with or without penetration techniques, or other routes of immunization. Priming by transcutaneous immunization (TCI) with either single or multiple applications may be followed with enteral, mucosal, transdermal, and/or other parenteral techniques for boosting immunization with the same or altered antigens. Priming by an enteral, mucosal, transdermal, and/or other parenteral route with either single or multiple applications may be followed with transcutaneous techniques for boosting immunization with the same or altered antigens. It should be noted that TCI is distinguished from conventional topical techniques like mucosal or transdermal immunization because the former (mucosal immunization) requires a mucous membrane (e.g., lung, mouth, nose, rectum) not found in the skin and the latter (transdermal immunization) requires perforation of the skin through the dermis. The formulation may include additional antigens such that application to skin induces an immune response to multiple antigens.

In addition to fusion proteins of the invention, the formulation may comprise a vehicle. For example, the formulation may comprise an AQUAPHOR, Freund, Ribi, or Syntex emulsion; water-in-oil emulsions (e.g., aqueous creams, ISA-720), oil-in-water emulsions (e.g., oily creams, ISA-51, MF59), microemulsions, anhydrous lipids and oil-in-water emulsions, other types of emulsions; gels, fats, waxes, oil, silicones, and humectants (e.g., glycerol).

An adjuvant conjugated or fused to fusion proteins of the invention or as part of the formulation is also contemplated. The choice of adjuvant may allow potentiation or modulation of the immune response. Moreover, selection of a suitable adjuvant may result in the preferential induction of a humoral or cellular immune response, specific antibody isotypes (e.g., IgM, IgD, IgA1, IgA2, IgE, IgG1, IgG2, IgG3, and/or IgG4), and/or specific T-cell subsets (e.g., CTL, Th1, Th2 and/or T_{DTH}). The adjuvant is preferably a chemically activated (e.g., proteolytically digested) or genetically activated (e.g., fusions, deletion or point mutants) ADP-ribosylating exotoxin or B subunit thereof. An activator of Langerhans cells may also be used as an adjuvant. Examples of such activators include proteins like chemokines, cytokines, differentiation factors, and growth factors (e.g., members of the TGFβ superfamily). Good manufacturing practices are known in the pharmaceutical industry and regulated by government agencies (e.g., Food and Drug Administration). A liquid formulation may be prepared by dissolving an intended component of the formulation in a sufficient amount of an appropriate solvent. Generally, dispersions are prepared by incorporating the various components of the formulation into a vehicle which contains the dispersion medium. For production of a solid form from a liquid formulation, solvent may be evaporated at room temperature or in an oven. Blowing a stream of nitrogen or air over the surface accelerates drying; alternatively, vacuum drying or freeze drying can be used.

The relative amounts of active ingredients within a dose and the dosing schedule may be adjusted appropriately for efficacious administration to a subject (e.g., animal or human). This adjustment may depend on the subject's particular disease or condition, and whether therapy or prophylaxis is intended. To simplify administration of the formulation to the subject, each unit dose would contain the active ingredients in predetermined amounts for a single round of immunization.

There are numerous causes of protein instability or degradation, including hydrolysis and denaturation. In the case of denaturation, the protein's conformation is disturbed and the protein may unfold from its usual globular structure. Rather than refolding to its natural conformation, hydrophobic interaction may cause clumping of molecules together (i.e., aggregation) or refolding to an unnatural conformation. Either of these results may entail diminution or loss of antigenic or adjuvant activity. Stabilizers may be added to lessen or prevent such problems.

The formulation, or any intermediate in its production, may be pretreated with protective agents (i.e., cryoprotectants and drying stabilizers) and then subjected to cooling rates and final temperatures that minimize ice crystal formation. By proper selection of cryoprotective agents and the use of preselected drying parameters, almost any formulation might be dried for a suitable desired end use.

It should be understood in the following discussion of optional additives like binders, buffers, colorings, dessicants, diluents, humectants, preservatives, and stabilizers are described by their function. Thus, a particular chemical may act as some combination of the aforementioned. Such chemicals would be considered immunologically-inactive because they do not directly induce an immune response, but they increase the response by enhancing immunological activity of the antigen or adjuvant: for example, by reducing modification of the antigen or adjuvant, or denaturation during drying and hydrating cycles.

Stabilizers include dextrans and dextrins; glycols, alkylene glycols, polyalkane glycols, and polyalkylene glycols, sugars and starches, and derivatives thereof are suitable. Preferred additives are nonreducing sugars and polyols. In particular, trehalose, hydroxymethyl or hydroxyethyl cellulose, ethylene or propylene glycol, trimethyl glycol, vinyl pyrrolidone, and polymers thereof may be added. Alkali metal salts, ammonium sulfate, magnesium chloride, and surfactants (e.g., nonionic detergent), may stabilize proteinaceous adjuvants or antigens; optionally adding a carrier (e.g., agar, albumin, gelatin, glycogen, heparin), and freeze drying may further enhance stability. A polypeptide may also be stabilized by contacting it with a sugar such as, for example, a monosaccharide, disaccharide, sugar alcohol, and mixtures thereof (e.g., arabinose, fructose, galactose, glucose, lactose, maltose, mannitol, mannose, sorbitol, sucrose, xylitol). Polyols may stabilize a polypeptide, and are water-miscible or water-soluble. Various other excipients may also stabilize polpeptides, including amino acids, fatty acids and phospholipids, metals, reducing agents, and metal chelating agents. The stabilizer may be between 0.1% (w/v) and 10% (w/v) or between 1% (w/v) and 5% (w/v) of the adhesive formulation.

Single-dose formulations can be stabilized in poly(lactic acid) (PLA) and poly (lactide-co-glycolide) (PLGA) microspheres by suitable choice of stabilizer or other excipients. Trehalose may be advantageously used as an additive because it is a nonreducing saccharide, and therefore does not cause aminocarbonyl reactions with substances bearing amino groups such as proteins. Although stabilizers like high concentrations of sugar will combat the growth of microbes like bacteria and fungi, preservatives are typically antimicrobial agents that actively eliminate (e.g., bactericidal) or reduce the growth of microbes (e.g., bacteriostatic). Antioxidants may also be used to prevent oxidation of active ingredients of the formulation.

It is conceivable that a formulation or patch that can be administered to the subject in a dry, nonliquid (i.e., solid) form, may allow storage in conditions that do not require a cold chain. An antigen may be mixed with a heterologous adjuvant, placed on a dressing to form a patch, and allowed to completely dry. This dry patch can then be placed on skin with the dressing in direct contact with the skin for a period of time and be held in place covered with an occlusive backing layer (e.g., plastic or wax film).

### Methods of Treatment

The invention provides methods to induce an immune response and/or to treat a subject (e.g., a human or animal in need of treatment such as prevention of disease, protection from effects of infection, therapy of existing disease or symptoms, or combinations thereof). Diseases other than infection include cancer, allergy, and autoimmunity. When the antigen is derived from a pathogen, the treatment may vaccinate the subject against infection by the pathogen or against its pathogenic effects such as those caused by toxin secretion. The invention may be used therapeutically to treat existing disease, protectively to prevent disease, to reduce the severity and/or duration of disease, to ameliorate symptoms of disease, or combinations thereof.

In one embodiment, the invention provides methods of inducing an immune response in a subject comprising application of bARE-STa fusion proteins. In another embodiment of the invention, the fusion proteins of the invention are an active ingredient in a patch and/or topical formulation.

In one embodiment, the invention provides methods of inducing an immune response in a subject comprising a bARE selected from the group consisting of (CT), heat-labile enterotoxin from E. coli (LT), Pseudomonas exotoxin A (ETA), pertussis toxin (PT), and diphtheria toxin (DT), or portions or fragments thereof. Other ADP-ribosylating toxins are also contemplated as part of the invention.

In another embodiment, the invention provides methods of inducing an immune response in a subject comprising application of different domains of a bacterial ADP ribosylating exotoxins fused to STa, or portions thereof. For example, it is contemplated that the A subunit, or portions thereof, of a bARE can be fused with STa, or portions thereof. In addition, it is contemplated that the B subunit, or portions thereof, of any bARE can be fused with STa. It is also it is contemplated that the whole bARE toxin (holotoxin) is fused with the STa. In a specific embodiment, the bARE is LT, or portions thereof, is fused to Sta (LT-STa). In another embodiment, the LT B subunit (LTB), or portions thereof, is fused to STa (LTB-STa). In another embodiment, the LT A subunit (LTA) is fused to STa (LTA-STa). In another embodiment the LT, LTA or LTB, or portions thereof, will be fused to the proSTa to create LT-proSTa, LTA-proSTa, or LTB-proSTa fusion proteins, respectively. It is also contemplated that fusion proteins of the invention may include a linker peptide between the fused portions to provide greater physical separation between the moieties. In one embodiment the linker is SEQ ID NO: 7 or SEQ ID NO: 9. It is also contemplated that all the above constructs can be used together or separately for transcutaneous immunizations.

The invention also contemplates a method of preventing a disease by applying a patch comprising bARE-STa fusions proteins. In one another embodiment, a method of preventing a disease by applying a patch comprising LT-STa fusions proteins is also contemplated. In a specific embodiment a method of preventing Traveler's diarrhea is contemplated.

The invention also contemplates a method of inducing an antigen-specific immune response comprising applying a formulation to an area of the skin of a subject thereby inducing an antigen-specific immune response, wherein the formulation comprises a fusion protein containing a bARE fused to a STa exotoxin.

The invention also provides methods of inducing an antigen specific immune response comprising applying a formulation to an area of the skin of a subject thereby inducing an antigen-specific immune response, wherein the formulation comprises a fusion protein containing bARE fused to STa or any of the fusion proteins of the invention. The method further includes treating an area of the skin prior to or concurrently with applying said formulation. The application site may be protected with anti-inflammatory corticosteroids such as hydrocortisone, triamcinolone and mometazone or nonsteroidal anti-inflammatory drugs (NSAID) to reduce possible local skin reaction or modulate the type of immune response. Similarly, anti-inflammatory steroids or NSAID may be included in the patch material, or liquid or solid formulations; and corticosteroids or NSAID may be applied after immunization. IL-10, TNFα, other immunomodulators may be used instead of the anti-inflammatory agents. Moreover, the formulation may be applied to skin overlying more than one draining lymph node field using either single or multiple applications. The formulation may include additional antigens such that application induces an immune response to multiple antigens. In such a case, the antigens may or may not be derived from the same source, but the antigens will have different chemical structures so as to induce an immune response specific for the different antigens. Multi-chambered patches could allow more effective delivery of multivalent vaccines as each chamber covers different antigen presenting cells. Thus, antigen presenting cells would encounter only one antigen (with or without adjuvant) and thus would eliminate antigenic competition and thereby enhancing the response to each individual antigen in the multivalent vaccine.

The invention also encompasses a method of treating, preventing, or inhibiting an enterotoxigenic Escherichia coli (ETEC) infection in a subject comprising applying to an area of the skin of said subject a therapeutically effective amount of a formulation comprising a fusion protein containing a bARE fused to a STa exotoxin, thereby inducing an antigen-specific immune response to treat, prevent, or inhibit an ETEC infection. The invention also encompasses a composition formulated for transcutaneous immunization containing an effective amount of a fusion protein comprising a bacterial ADP-ribosylating endotoxin fused to a STa endotoxin for inducing an antigen-specific immune response by epicutaneous administration.

Other Traveler's diseases of interest that can be treated. Include are campylobacteriosis (Campylobacter jejum), giardiasis (Giardia intestinalis), hepatitis (hepatitis virus A or B), malaria (Plasmodium falciparum, P. vivax, P. ovale, and P. malariae), shigellosis (Shigella boydii, S. dysenteriae, S. flexneri, and S. sonnei), viral gastroenteritis (rotavirus), and combinations thereof. Effectiveness may be assessed by clinical or laboratory criteria.

The formulation of the active ingredient in a patch or other formulation or composition which will be effective in the treatment, prevention or management of a disease (such as Traveler's disease) can be determined by standard research techniques. For example, the dosage of the composition which will be effective in the treatment, prevention or management of specific diseases can be determined by administering the composition to an animal model such as, e.g., the animal models disclosed herein or known to those skilled in the art. In addition, in vitro assays may optionally be employed to help identify optimal dosage ranges.

Selection of the preferred effective dose can be determined (e.g., via clinical trials) by a skilled artisan based upon the consideration of several factors which will be known to one of ordinary skill in the art. Such factors include the disease to be treated or prevented, the symptoms involved, the patient's body mass, the patient's immune status and other factors known by the skilled artisan to reflect the accuracy of administered pharmaceutical compositions. Effective doses may be extrapolated from dose-response curves derived from in vitro or animal model test systems

Undesirable properties or harmful side effects (e.g., allergic or hypersensitive reaction; atopy, contact dermatitis, or eczema; systemic toxicity) may be reduced by modification without destroying its effectiveness in transcutaneous immunization. Modification may involve, for example, removal of a reversible chemical modification (e.g., proteolysis) or encapsulation in a coating which reversibly isolates one or more components of the formulation from the immune system. For example, one or more components of the formulation may be encapsulated in a particle for delivery (e.g., microspheres, nanoparticles) although we have shown that encapsulation in lipid vesicles is not required for transcutaneous immunization and appears to have a negative effect. Phagocytosis of a particle may, by itself, enhance activation of an antigen presenting cell by upregulating expression of MHC Class I and/or Class II molecules and/or costimulatory molecules (e.g., CD40, B7 family members like CD80 and CD86). Alternative methods of upregulating such molecules by activating an antigen presenting cell are also known (see above).

Transcutaneous delivery of the formulation may target Langerhans cells and, thus, achieve effective and efficient immunization. Cells are found in abundance in the skin and are efficient antigen presenting cells (APC), which can lead to T-cell memory and potent immune responses. Because of the presence of large numbers of Langerhans cells in the skin, the efficiency of transcutaneous delivery may be related to the surface area exposed to antigen and adjuvant. In fact, the reason that transcutaneous immunization is so efficient may be that it targets a larger number of these efficient antigen presenting cells than intramuscular immunization.

Immunization may be achieved using epicutaneous application of a simple formulation of antigen and adjuvant, optionally covered by an occlusive dressing or using other patch technologies, to intact skin with or without chemical or physical penetration. Transcutaneous immunization according to the invention may provide a method whereby antigens and adjuvant can be delivered to the immune system, especially specialized antigen presentation cells underlying the skin (e.g., dendritic cells like Langerhans cells). The patch may be worn for as briefly as 30 sec; 1 min to 5 min; or less than 1 hour, 2 hours, 4 hours, 6 hours, 8 hours, 12 hours, 15 hours, 18 hours, 24 hours, or 48 hours. In contrast to transdermal patches delivering drugs, the release characteristics of the patch of the invention does not need to be constant or prolonged. It is preferred that the immunologically-active protein may be released quickly and quantitatively.

Without further description, it is believed that one of ordinary skill in the art can, using the preceding description and the following illustrative examples, make and utilize the claimed invention. The following working examples therefore, specifically point out preferred embodiments of the present invention, and are not to be construed as limiting in any way the remainder of the disclosure. All articles, publications, patents and documents referred to throughout this application are hereby incorporated herein by reference in their entirety.

### EXAMPLES

### Example 1

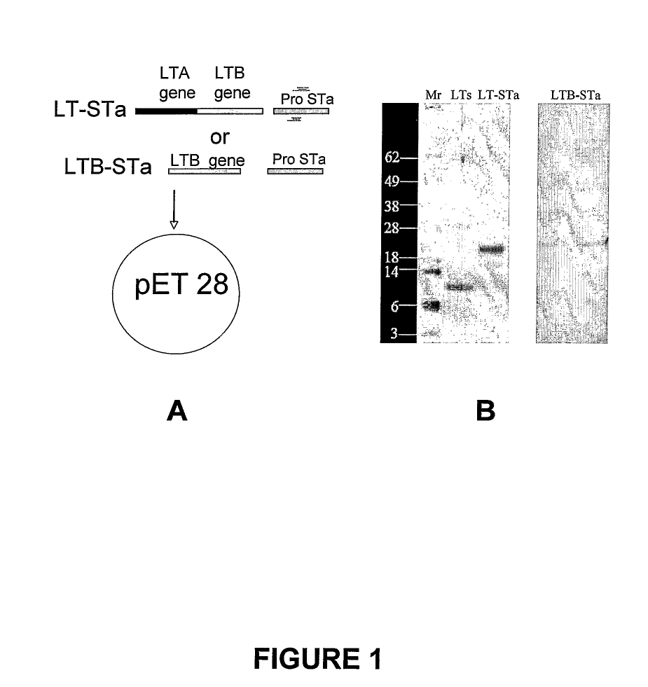

Construction of LT/pro-STα and LTB/pro-STα fusion proteins. The LT gene (1148 bp), LT-B (378 bp) and the human STa gene (159 bp) were amplified from genomic DNA of the ETEC H10407 strain (for example, ATCC accession no. 35401; other strains are optionally used in the practice of the methods of the invention) using the 5′ and 3′ primers listed in Table 1. The forward primer created a unique Nco1 site and the reverse primer created a BamH1 site on LT and LTB gene. BamH1 and Xho1 restriction sites were created at N-terminal and C-terminal of the STa gene. FIG. 1A. The genes were purified from agarose gels. The expression vector pET28 was digested with Nco1 and Xho1 restriction enzymes. The STa gene was ligated for 3 h at room temperature to the LTAB genes or LTB and the fusion gene was cloned into the pET28 vector. Competent E. coli Mach-1 cells were transformed with the ligation mixture and recombinants selected by growth on LB agar containing 50 μg/ml kanamycin. Preliminary screening was done by PCR amplification of the fusion genes from the colonies using LT or LTB forward primer and the STa reverse primer. Subsequently the plasmid was digested with Nco1/Xho1 and the fusion genes were recovered. The positive plasmid was transformed into BL21 (DE3) for expression. The junction of the gene fusions of LT or LTB and STa carried in pET28 were determined by dye terminator cycle sequence (Veritas, Inc).

An alternative strategy is to fuse processed STa (19 amino acids) to LTB. This strategy used a seven amino acid linker between the LTB-subunit and STa. In this instance assembly of the B-pentamer was perturbed. However, this fusion protein was immunogenic in that antibodies produced against STa were capable of neutralizing native ST.

TABLE 1

Forward and reverse primers
used to clone LT and STa

| | |
|---|---|
| Seq ID | Primer design |

| | | |
|---|---|---|
| 1 | LTA5′ | GGCCCATGGATGAAAAATATAACTACTTTCATT |
| 2 | LTB5′ | GGCCCATGGATGAATAAAGTAAAAGTTAT |
| 3 | LTB3′ | GGCGGATCCGTTTTCCATACTGATTGC |
| 4 | STa5′ | GGCGGATCCCAGGATGCTAAACCAGTAGAG |
| 5 | STa3′ | GGCCTCGAGCTAATAGCACCCGGTACAAGC |

### Example 2

Purification of the LTB-proSTα and LT-proSTα. LTB-proSTa and LT-proSTa fusion proteins were purified from E. coli BL21 by affinity chromatography using immobile D-galactose. The recombinant bacteria were cultured in LB broth containing 50 μg/ml of kanomycin and cultures were grown overnight at 37° C. The next day 1:10 diluted culture were inoculated into LB medium at 37° C. and grown to 0.5˜0.7 at OD₆₀₀. Cultures were induced with 0.5 mM IPTG for 3 hr. The cells were harvested by centrifugation at 6000 rpm and cells were suspended in TEAN buffer (0.05 M Tris, 0,001 M EDTA, 0.2 M NaC1 at pH 7.5) and lysed by sonication. The crude lysate was clarified by centrifugation twice. The supernate was directly applied to an immobilized galactose column. The column was washed extensively with TEAN buffer. The fusion proteins were eluted with TEAN buffer containing 0.3 M galactose. LT-proSTa and LTB-proSTa were found to bind to the D-galactose affinity column, indicating that the STa did not interfere with the assembly of the B-pentamer. The expression level of LTB-proSTa is low and this will require additional work to improve expression.

A GM-1 ELISA method was used to determine if LT-proSTa binds to the GM1 ganglioside receptor. This was done by coating 96 well plates with 1 μg GM 1 ganglioside and adding serially diluted affinity purified fusion protein to the wells. A goat anti-LTB specific antibody was used to detect LT-proSTa and an alkaline phosphatase conjugated rabbit anti-goat IgG antibody was used to determine if LT-proSTa associated with the ganglioside. Our results indicated that the fusion specifically associated with GM 1 ganglioside, indicating the B-subunit was assembled.

LT-proSTa was characterized by SDS-PAGE and size exclusion (SE) HPLC. Examination of FIG. 1B shows the migration of the LTA and LTB subunits using a reference standard (Lane 2). Lane 3 reveals that the fusion protein migrates at a higher molecular weight than does the B-subunit. The apparent molecular weight of the fusion protein is approximately 20,000 Daltons, as expected for the fusion. There is an apparent absence of the A-subunit with the fusion protein. Western blot analysis of the soluble cell lysate with a LTA specific monoclonal antibody shows the LTA subunit is abundantly expressed by the cell. These observations indicate that fusing proSTa to LTB does not interfere with the assembly of the B pentamer, but it does interfere with A-subunit association with the LTB-STa fusion to form the holotoxin. For the purpose of clarity, this fusion protein is referred to as LT-proSTa.

### Example 3

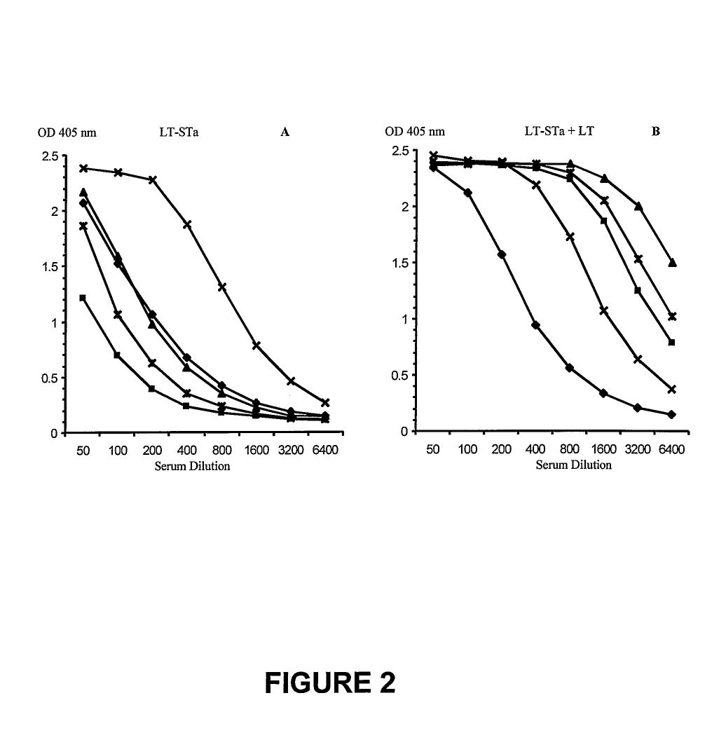

Mice were anesthetized and the dorsal caudal surface at the base of the tail was shaved prior to patch application. The shaved skin was hydrated with saline and pretreated with emery paper to disrupt the stratum corneum. A gauze pad on an adhesive backing was loaded (25 μl) with 25 μg LT-STa fusion protein alone or mixed with 10 μg LT. Patches were applied for 18 hr. All mice were immunized on day 0 and 14 and serum was collected two weeks after the second immunization. An ELISA method was used to detect serum antibodies to STa. FIG. 2 represents titration curves for individual animals.

### Example 4

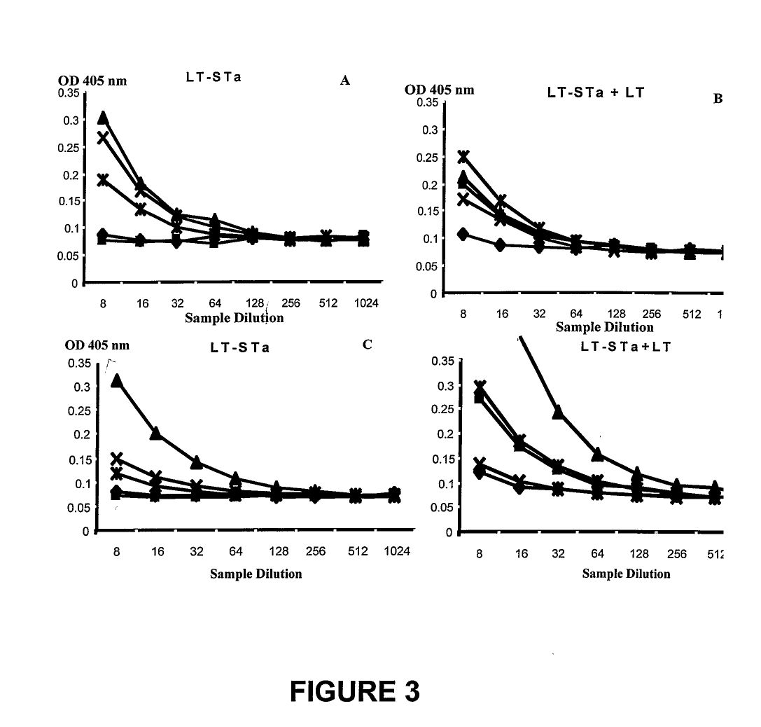

Mice were prepared for immunization as described in example 3. All mice were immunized with three doses of LT-STa alone (25 μg) or mixed with LT holotoxin (10 μg) on day 0, 14 and 28. Fresh fecal samples were collected from individual animals seven days after the third immunization. The samples were homogenized and the clarified extract was assayed for STa-specific IgA (panels A and B) and STa-specific IgG (panels C and D) using an ELISA method. FIGS. 3 A-D represents titration curves for individual animals.

### Example 5

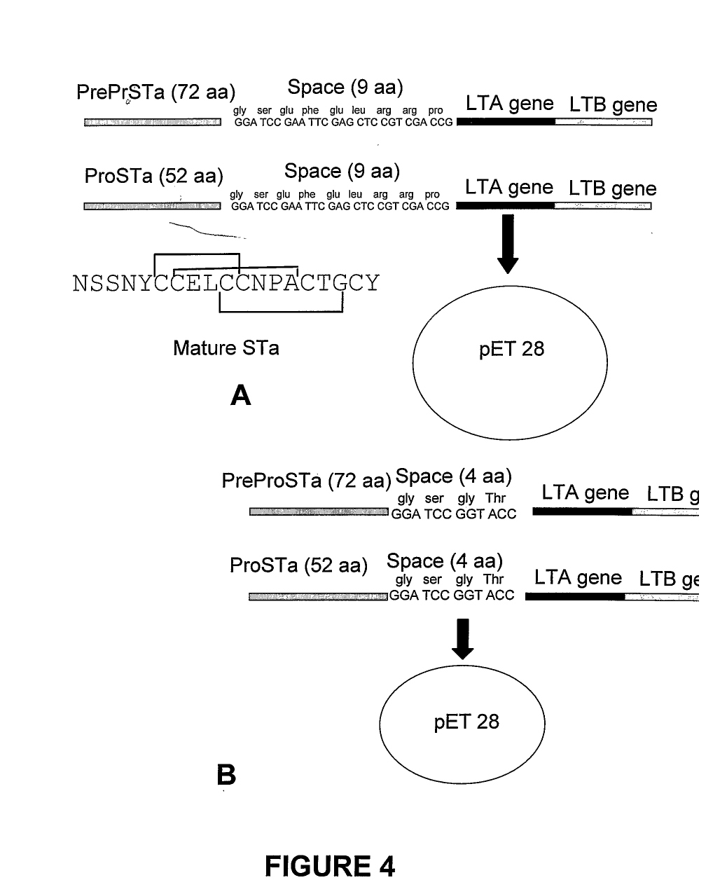

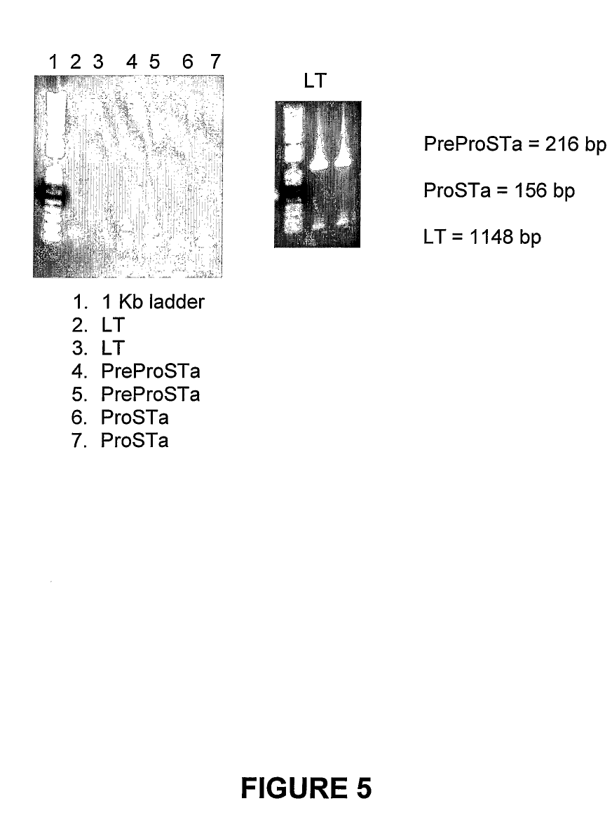

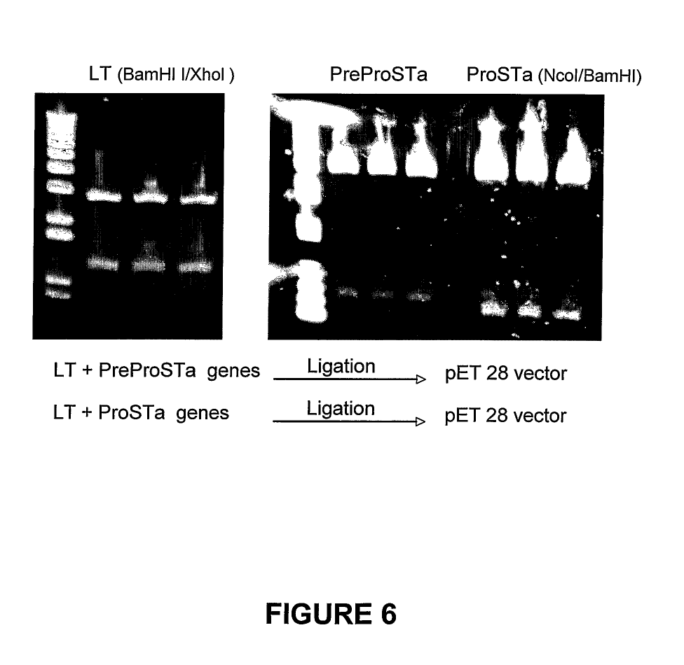

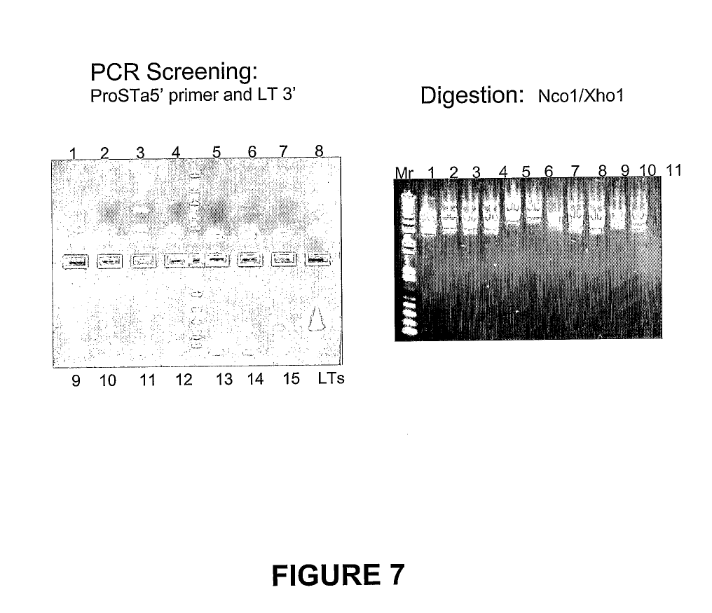

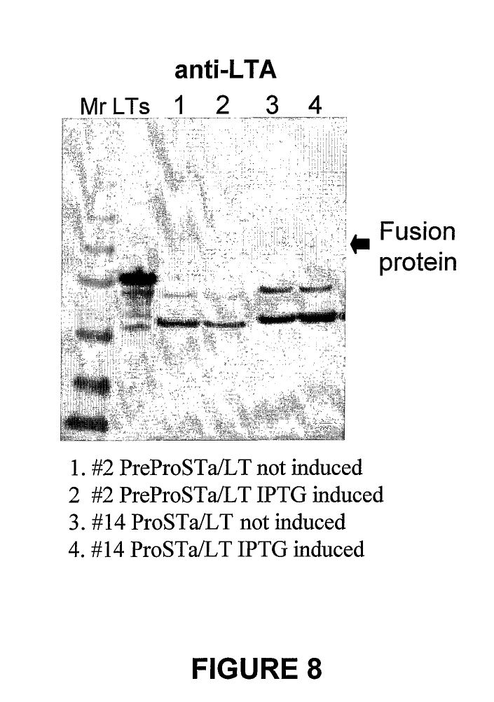

Construction of prepro-STa and pro-STa fused to the LTA subunit with intervening spacer sequences. FIG. 4 (A) shows the 9 mer (gly-ser-glu-phe-glu-leu-arg-arg-pro) (SEQ ID NO: 7) and (B) 4 mer (gly-ser-gly-thr) (SEQ ID NNO: 9) spacer sequences placed between the Prepro-STa and pro-STa and fused to the N-terminus of LTA. pET28 was digested with NcoI/Xho1. PCR was used to amplify LT and STa genes from H10407 genomic DNA. The LT gene was digested with BamHI at 5′ and Xho1 at 3′ end. The STa gene was digested with NcoI at 5′ and BamHI at 3′ end. The restricted LT and STa genes were ligated with the digested vector. The ligation mixture was then transformed into Mach1 cell. Mini-preps and analysis of inserts by digestion and PCR was used to identify correct constructs (FIGS. 5, 6 and 7). The correct construct was transformed into BL21 for expression. A western blot analysis confirms that the correct construction and expression of prepro-STa and pro-STa fused to the LTA (FIG. 8).

It should be understood that the foregoing discussion and examples merely present a detailed description of certain preferred embodiments. It therefore should be apparent to those of ordinary skill in the art that various modifications and equivalents can be made without departing from the spirit and scope of the invention. All journal articles, other references, patents, and patent applications that are discussed herein are incorporated by reference herein their entirety.

1: A fusion protein comprising a bacterial ADP-ribosylating exotoxin (bARE) fused to a STa exotoxin, wherein said exotoxins are fused via a peptide linker.

2: A composition formulated for transcutaneous immunization containing an effective amount of a fusion protein comprising a bacterial ADP-ribosylating exotoxin fused to a STa exotoxin via a peptide linker for inducing an antigen-specific immune response by epicutaneous administration.

3: The fusion protein of claim 1, wherein the peptide linker has the amino acid sequence as set forth in SEQ ID NO: 7 or SEQ ID NO: 9.

4: A patch for transcutaneous immunization comprising a fusion protein, wherein said fusion protein comprises a bacterial ADP-ribosylating exotoxin (bARE) fused to a STa exotoxin.

5: The patch of claim 4, wherein said fusion protein further comprises a peptide linker.

6: The patch of claim 5, wherein the peptide linker has the amino acid sequence as set forth in SEQ ID NO: 7 or SEQ ID NO: 9.

7: The patch of claim 4, wherein the STa exotoxin is a pro-STa exotoxin.

8: The patch of claim 4, wherein the STa exotoxin is linked to SEQ ID NO: 7.

9: The patch of claim 4, wherein said bARE is selected from the group consisting of a cholera toxin (CT), heat-labile enterotoxin from E. coli (LT), Pseudomonas exotoxin A (ETA), pertussis toxin (PT) and diphtheria toxin (DT).

10: The patch of claim 9, wherein said bARE is LT.

11: The patch of claim 4, wherein said bARE comprises the A subunit of a bARE.

12: The patch of claim 11, wherein said A subunit of bARE is selected from the group consisting of a cholera toxin A subunit (CTA), heat-labile enterotoxin A subunit from E. coli (LTA), Pseudomonas exotoxin A-A subunit (ETAA), pertussis toxin A subunit (PTA) and diphtheria toxin A subunit (DTA).

13: The patch of claim 11, wherein said A subunit of bARE is LTA.

14: The patch of claim 4, wherein said bARE comprises the B subunit of a bARE.

15: The patch of claim 14, wherein said B subunit of bARE is selected is selected from the group consisting of a cholera toxin B subunit (CTB), heat-labile enterotoxin B subunit from E. coli (LTB), Pseudomonas exotoxin A-B subunit (ETAB), pertussis toxin B subunit (PTB) and diphtheria toxin B subunit (DTB).

16: The patch of claim 14, wherein said B subunit of bARE is LTB.

17: The patch of claim 4, wherein said bARE comprises the bARE holotoxin.

18: The patch of claim 17, wherein said bARE holotoxin is selected is selected from the group consisting of a cholera toxin (CT-holo), heat-labile enterotoxin from E. coli (LT-holo), Pseudomonas exotoxin A (ETA-holo), pertussis toxin (PT-holo) and diphtheria toxin (DT-holo).

19: The patch of claim 17, wherein said bARE holotoxin is LT-holo.

20: A method of inducing an antigen-specific immune response in a subject comprising applying the patch of claim 4 to said subject to induce an antigen-specific immune response.

21: A method of preventing a disease in a subject comprising applying the patch of claim 4 to said subject to induce an antigen-specific immune response, thereby preventing a disease.

22: A method of claim 21, wherein said disease is traveller's diarrhea.

23: A method of inducing an antigen-specific immune response comprising applying a formulation to an area of the skin of a subject thereby inducing an antigen-specific immune response, wherein said formulation comprises a fusion protein containing a bARE fused to a STa exotoxin.

24: The method of claim 23, wherein said fusion protein further comprises a peptide linker.

25: The method of claim 24, wherein the peptide linker has the amino acid sequence as set forth in SEQ ID NO: 7 or SEQ ID NO: 9.

26: The method of claim 23, wherein the STa exotoxin is a pro-STa exotoxin.

27: The method of claim 23, wherein the STa exotoxin is linked to SEQ ID NO: 9.

28: The method of claim 17, wherein said bARE is selected from the group consisting of a cholera toxin (CT), heat-labile enterotoxin from E. Coli (LT), Pseudomonas exotoxin A (ETA), pertussis toxin (PT) and diphtheria toxin (DT).

29: The method of claim 28, wherein said bARE is LT.

30: The method of claim 23, wherein said bARE comprises the A subunit of a bARE.

31: The method of claim 30, wherein said A subunit of bARE is selected is selected from the group consisting of a cholera toxin A subunit (CTA), heat-labile enterotoxin A subunit from E. coli (LTA), Pseudomonas exotoxin A-A subunit (ETAA), pertussis toxin A subunit (PTA) and diphtheria toxin A subunit (DTA).

32: The method of claim 31, wherein said A subunit of bARE is LTA.

33: The method of claim 23, wherein said method further comprises treating said area of the skin to enhance said immune response.

34: The method of claim 33, wherein treating said area of the skin is prior to or concurrently with applying said formulation.

35: A method of treating, preventing, or inhibiting an enterotoxigenic Escherichia coli (ETEC) infection in a subject comprising applying to an area of the skin of said subject a therapeutically effective amount of a formulation comprising a fusion protein containing a bARE fused to a STa exotoxin, thereby inducing an antigen-specific immune response to treat, prevent, or inhibit an ETEC infection.

36: A patch for transcutaneous immunizations comprising a fusion protein, wherein said fusion protein comprises a bacterial ADP-ribosylating exotoxin (bARE) B subunit fused to a STa exotoxin.

37: The patch of claim 36, wherein said B subunit of a bARE is selected is selected from the group consisting of a cholera toxin B subunit (CTB), heat-labile enterotoxin B subunit from E. coli (LTB), Pseudomonas exotoxin A-B subunit (ETAB), pertussis toxin B subunit (PTB) and diphtheria toxin B subunit (DTB).

38: The patch of claim 37, wherein said B subunit of bARE is LTB.

claim 39: The patch of claim 36, wherein said fusion protein further comprises a peptide linker.

40: The patch of claim 39, wherein the peptide linker has the amino acid sequence as set forth in SEQ ID NO: 7 or SEQ ID NO: 9.
